(19) United States
(12) Patent Application Publication
(10) Pub. No.: US 2026/0185962 A1
(43) Pub. Date: Jul. 2, 2026

(54) PARTICLE MOTION TRACKING FOR DETECTION IN BIOMOLECULE SEPARATION

(71) Applicant: Wayne State University, Detroit, MI (US)

(72) Inventor: Thomas H. Linz, Livonia, MI (US)

(73) Assignee: Wayne State University, Detroit, MI (US)

(21) Appl. No.: 19/431,855

(22) Filed: Dec. 23, 2025

Related U.S. Application Data
(60) Provisional application No. 63/740,301, filed on Dec. 30, 2024.

(51) Int. Cl. G01N 27/447; B01L 3/00; G01N 33/68

(52) U.S. Cl. CPC G01N 27/44721; B01L 3/502761; G01N 27/44791; G01N 33/6803; B01L 2300/069; B01L 2300/0861; B01L 2400/0421

(57) ABSTRACT

Described herein are systems that track positions of particles embedded in a gel within a microfluidic channel. As biomolecules separate through the gel, they collide with the tracking particles and deform the gel, which alters particle motion. Tracking the position and instantaneous displacement of the nanoparticles enables detection and quantitation of biomolecule concentrations in the sample. The described platform provides high sensitivity detection of biomolecules while minimizing or obviating time- and cost-intensive preparation steps.

### CROSS-REFERENCE TO RELATED APPLICATION

This application claims priority to and the benefit of the earlier filing of U.S. Provisional Application No. 63/740,301, filed on Dec. 30, 2024, which is incorporated by reference herein in its entirety.

### STATEMENT REGARDING FEDERALLY SPONSORED RESEARCH OR DEVELOPMENT

This invention was made with government support under contract R35GM150518 awarded by the National Institutes of Health. The government has certain rights in the invention.

### FIELD OF THE DISCLOSURE

The present disclosure relates generally to methods and devices for separating molecules, such as biomolecules, based at least in part on detecting motion of particles other than the biomolecules embedded in a separation medium.

### SUMMARY OF THE DISCLOSURE

Described herein is a system that tracks the position of nanoparticles embedded in a gel within a microfluidic channel. As biomolecules separate through the gel, they collide with the nanoparticles and locally deform the gel, which alters particle motion. Tracking the position and instantaneous displacement of the nanoparticles enables quantitation of biomolecule concentrations in the sample. Proof-of-concept demonstrations are provided for the analysis of a biomedically relevant sugar used in drug delivery, a therapeutic peptide, and a small molecule. The described platform provides high sensitivity detection of biomolecules while minimizing or obviating time- and cost-intensive preparation steps.

This report describes the development of a separations-based sensor that integrates particle motion tracking (PMT) into thermal gel electrophoresis (TGE) (TGE-PMT) for label-free quantitation of biomolecules, such as polysaccharides, peptides, and the like. In the context of this document, “label-free” refers to there not being a label attached to the biomolecule(s); as exemplified, label(s) may be included in other components in the analysis system. Tracking nanoparticles and analytes were spiked into liquid-phase thermal gel that was then loaded throughout a single-channel microfluidic device (FIG. 1). The thermal gel was solidified to immobilize the particles in a lattice of packed gel micelles within the channel. Voltage was applied to induce electrokinetic enrichment and separation of the analytes (exemplified by biologically relevant sugars). As the bands of analyte collided with the nanoparticles, the baseline motion of the nanoparticles was altered (FIG. 2) to provide a quantitative response. The studies herein demonstrate feasibility for integrating PMT with TGE and establish best practices for quantitative data processing. The described approach obviated the need to label sugars and perform subsequent washing and purification steps. This label-free analysis can provide universal detection of diverse biomolecules in complex biomedical samples.

Also provided are embodiments in which the electrophoresis device includes reference markings or reference particles that can be used to correct data for movement of the device itself. This enables collection and analysis of data reflecting only the movement of nanoparticle markers embedded in the gel, which is provided by the impact of collisions with biomolecular analytes migrating through the gel/device in an electric field.

The current disclosure provides an improved method of analyte detection and/or quantification in gel electrophoresis, the improvement including: including tracking particles within the gel; and indirectly detecting a wavefront (band) of analyte migrating within the gel, which migrating is caused at least in part by analyte interaction with an applied electric field, the indirect detecting including detecting movement of the tracking particles in response to collisions with analyte molecules in the wavefront (band) and/or deformations of the gel. In examples of this method, the tracking particles are incapable of interacting with the analyte by binding, adsorption, or chemical reaction. Instead, the tracking particles interact through collision, such as elastic or partially elastic collision.

In example of the method embodiments herein, the tracking particles have an average diameter less than 10 microns. For instance, in embodiments the tracking particles have an average diameter selected from: ≤8 microns, ≤5 microns, ≤2 microns, ≤1 micron, ≤900 nanometers, ≤800 nanometers, ≤750 nanometers, ≤700 nanometers, ≤600 nanometers, ≤500 nanometers, ≤400 nanometers, ≤300 nanometers, ≤200 nanometers, ≤100 nanometers, ≤80 nanometers, ≤75 nanometers, ≤50 nanometers, ≤40 nanometers, 100 nanometers to 2 microns, 100 nanometers to 1 micron, 100-800 nanometers, 100-600 nanometers, 100-500 nanometers, 100-400 nanometers, 100-300 nanometers, 100-250 nanometers, 150-300 nanometers, 150-250 nanometers, 200-300 nanometers, 50-300 nanometers, 50-250 nanometers, 50-300 nanometers, 50-250 nanometers, 15-300 nanometers, 15-250 nanometers, 15-300 nanometers, 15-250 nanometers, 15 nanometers, 20 nanometers, 25 nanometers, 40 nanometers, 50 nanometers, 75 nanometers, 100 nanometers, 150 nanometers, 200 nanometers, 250 nanometers, 300 nanometers, 350 nanometers, 400 nanometers, 500 nanometers, 600 nanometers, 750 nanometers, 800 nanometers, 1 micron, or 2 microns. Specifically contemplated are embodiments of methods that employ at least two different defined-size tracking particle populations within the gel. Optionally, the average diameter of the two or more populations do not overlap, and optionally they are populations readily distinguishable using detection methods as provided herein.

In any of the provided method embodiments, detecting movement of the tracking particles can include determining displacement in an X dimension of at least one tracking particle from a time zero position, where the “x dimension” is parallel to the direction of migration of the analyte wavefront. Optionally, tracking particle displacement is calculated based on step size analysis (from one image frame to the next).

Any of the provided method embodiments can include determining the displacement of at least one, at least 3, at least five, at least seven, at least ten, at least 100, at least 1000, at least 10,000, or more than 10,000 tracking particles.

Though exemplified using a single or two defined analytes herein as well as in Cornejo & Linz (ACS Sensors 10:204-212, 2025), it is contemplated that the provided method embodiments are useful for the analysis of mixed analyte samples. Therefore, the method embodiments include examples that involve indirectly detecting migration of more than one analyte (including analytes in a mixed sample), each analyte having a wavefront (band) of migration distinguishable at least in part from the wavefront (band) of at least one other analyte(s) migrating in the gel.

Another embodiment is a method of label-free injectionless gel electrophoresis, the method including: providing a microfluidic device having a channel, the channel having a first end and a second end, the microfluidic device having a first reservoir coupled to the first end of the channel and a second reservoir coupled to the second end of the channel; loading into the channel of the microfluidic device a substantially homogenous mixture including: a mixed analyte sample; a plurality of tracking particles (for instance, nanoparticles, microparticles, or quantum dots); and a gel solution; providing a first reservoir solution in the first reservoir; providing a second reservoir solution in the second reservoir; and applying an electric field across the microfluidic device.

In any of the provided method embodiments, the tracking particles can include one or more of a polymer, a metal (such as gold or silver), silica or glass, a magnetic material, or a lipid. By way of example, tracking particles that include a polymer can include a dextran, a polystyrene, an acrylate, a poly(ethylene glycol) (PEG), a peptide, or a nucleic acid.

Also contemplated are tracking particles that are fluorescent or otherwise detectable particles. In other embodiments, the tracking particles include a detectable label, such as a dye, fluorophore, or other art-recognized label moiety.

With regard to operation of provided methods, the first reservoir solution includes a first electrolyte (at least one electrolyte) and the second reservoir solution includes a second electrolyte (at least one electrolyte). Optionally, the microfluidic device further includes a first electrode arranged in the first reservoir and a second electrode arranged in the second reservoir. By way of example, provided methods include anionic analytes migrating from the first reservoir to the second reservoir, and: the first electrode is a cathodic electrode; the first reservoir solution is a cathodic reservoir solution; the first reservoir is a cathodic reservoir; the second electrode is an anodic electrode; the second reservoir solution is an anodic reservoir solution; and the second reservoir is an anodic reservoir. In some example embodiments, the method includes cationic analytes migrating from the first reservoir to the second reservoir, and: the first electrode is an anodic electrode; the first reservoir solution is an anodic reservoir solution; the first reservoir is an anodic reservoir; the second electrode is a cathodic electrode; the second reservoir solution is a cathodic reservoir solution; and the second reservoir is a cathodic reservoir.

It is contemplated the methods can be used to analyze mixed analytes that include biomolecules. For instance, the mixed analyte sample will in various embodiments include at least one of nucleic acids, carbohydrates, peptides, or proteins. Methods that analyze mixtures of two or more of these types of these biomolecules are also provided.

Method embodiments also can further include solidifying the gel solution. By way of example, the gel is a sieving gel for resolving analytes in the sample. Optionally, the gel is thermally responsive. In examples, the gel employed in any of the described methods includes one or more of: agarose, polyacrylamide, a polymer sieving matrix, a capillary gel electrophoresis matrix, or a thermal gel polymer. Specifically contemplated thermal gel polymers include one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

Optionally, a buffer can be included in the gel solution and/or the mixed analyte sample.

Methods also provide detecting separation of analytes of the mixed analyte sample in the channel.

In any of the provided methods, optionally reference particles are embedded into or attached on at least a portion of the microfluidic device, for instance adjacent to the channel. The placement of reference particles is selected to allow imaging/detection of the reference particles concurrently with imaging/detection of tracking particles, such that movement of reference particles can be used in order to control for drift or device movement.

In any of the provided methods, the method can include indirectly detecting a wavefront of analyte migrating within the gel, which migrating results at least in part from analyte interaction with an electric field, by detecting movement of tracking particles in response to collisions of analyte molecules in the wavefront with the tracking particles and/or deformations of the gel. By way of example, the migrating (of analyte(s)) results at least in part from analyte interaction with an applied electric current and/or an applied electric voltage.

Also provided are microfluidic devices that are useful in methods provided herein. Provided microfluidic devices, in embodiments, include a channel, configured to accommodate a mixed analyte sample (e.g., containing at least one of nucleic acids, carbohydrates, peptides, or proteins) mixed with a gel solution, the channel having a first end and a second end; embedded within (or otherwise attached to) at least a portion of a wall of the channel, reference particles; a first reservoir coupled to the first end of the channel, the first reservoir being configured to accommodate a first reservoir solution; a second reservoir coupled to the second of the channel, the second reservoir being configured to accommodate a second reservoir solution; a first electrode arranged in the first reservoir; and a second electrode arranged in the second reservoir; wherein the first electrode and the second electrode are configured to apply an electric field across the microfluidic device.

Examples of such devices are configured for anionic analytes to migrate from the first reservoir to the second reservoir, and: the first electrode is a cathodic electrode; the first reservoir solution is a cathodic reservoir solution; the first reservoir is a cathodic reservoir; the second electrode is an anodic electrode; the second reservoir solution is an anodic reservoir solution; and the second reservoir is an anodic reservoir.

Additional examples of such devices are configured for cationic analytes to migrate from the first reservoir to the second reservoir, and: the first electrode is an anodic electrode; the first reservoir solution is an anodic reservoir solution; the first reservoir is an anodic reservoir; the second electrode is a cathodic electrode; the second reservoir solution is a cathodic reservoir solution; and the second reservoir is a cathodic reservoir.

In any of the provided device embodiments, the gel may be a sieving gel for resolving the sample. Optionally, the gel is thermally responsive. In examples, the gel employed in any of the described methods includes one or more of: agarose, polyacrylamide, a polymer sieving matrix, a capillary gel electrophoresis matrix, or a thermal gel polymer. Specifically contemplated thermal gel polymers include one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

In embodiments of the provided devices, the reference particles are configured as device movement/drift control reference markers.

Also provided are methods of assaying analyte migration in a liquid contained in a device (which liquid is optionally contained at least in part within a solidified gel), the method including: contacting the migrating analyte with a plurality of tracking particles of average diameter less than 10 microns, the particles being incapable of interacting with the analyte by binding, adsorption, or chemical reaction; observing motion of some or all of the tracking particles by at least one optical, fluorescence, or other electromagnetic measurement technique; and using differing motion of the tracking particles to infer presence and/or concentration of the migrating analyte.

In examples of such embodiments, the observing includes one or more of observing fluorescence, fluorescence lifetime, resonance energy transfer, phosphorescence, reflection, polarization, scattering, absorbance, chemiluminescence, or magnetic properties of some or all of the tracking particles. Optionally, the methods further including detection of electromagnetic emission (such as from detectable or labeled tracking particles and/or reference particles) at more than one wavelength.

The provided methods may further including particle tracking, single-particle tracking or tethered-particle motion tracking.

In embodiments of the methods, the analyte(s) migrating in the liquid includes electrophoretic, dielectrophoretic, isotachophoretic, or sedimentation motion. In specific examples, the analyte(s) migrating in the liquid includes electrophoresis, including through a gel.

In examples, the gel is a sieving gel for resolving migrating analytes. By way of example, the gel is thermally responsive. Specifically contemplated are embodiments in which the gel includes one or more of: agarose, polyacrylamide, a polymer sieving matrix, a capillary gel electrophoresis matrix, or a thermal gel polymer. Where the gel includes a thermal gel polymer, the polymer may include one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

The provided methods can further include observing tracking particles by camera, digital camera, PMT, scanner, microscope, telescope, detector array, time-gated, chopped, frequency-modulated, wavelength-filtered, polarization-sensitive, Raman, Surface-enhanced Raman, high numerical aperture, color-sensitive, lifetime, FRET, FRAP, intensified, phosphorescence, resistivity, ellipsometer, or high-density CCD observation, in flow, on a surface, or in suspension.

In examples of the methods, the tracking particles include nanoparticles, microparticles, or quantum dots—or mixtures of two or more thereof. Specifically contemplated are instances where two (or more) different size of tracking particles are used in the same method.

Tracking particles useful in provided methods can include one or more of a polymer, a metal (such as gold or silver), silica or glass, a magnetic material, or a lipid. Where the tracking particles include a polymer, they can include a dextran, a polystyrene, an acrylate, a poly(ethylene glycol) (PEG), a peptide, or a nucleic acid. Mixtures are also contemplated.

Optionally, the method involves controlling and/or monitoring a temperature of one or more elements of a system including the device used in the method.

Also provided are methods that further include assaying more than one migrating analyte, which plurality of migrating analytes are separated by the migration, and the method includes using differing motion of the tracking particles to infer presence and/or concentration of the more than one migrating analyte.

Also provided are methods that further include carrying out the analyte migrating assay in a device in including reference particles substantially equivalent in size and detection capability to the tracking particles with which the analyte(s) is contacted, wherein the method further includes controlling for device movement when calculating the differing motion of the tracking particles by detecting movement of one or more reference particles.

Yet another embodiment is an improved method of thermal gel electrophoresis (TGE), the improvement including: employing particle motion tracking (PMT) to detect and/or quantify label-free analyte(s) being subject to the TGE.

Also provided are label-free analyte separation strategies based on gel electrophoresis, and on particle motion tracking, substantially as disclosed herein.

Another embodiment is a method for separating and detecting and optionally quantifying two or more biomolecule species in a mixed sample, the method including thermal gel electrophoresis and particle motion tracking substantially as described herein.

Another example embodiment is a method of improving analyte detection in a gel electrophoresis device, including embedding or otherwise attaching reference particles in/on the gel electrophoresis device. By way of example, the embedded/attached reference particles in such methods are used to correct for device drift and/or to improve signal quality of movement of tracking particles used in the method.

### BRIEF DESCRIPTION OF THE DRAWINGS

Some of the drawings submitted herein may be better understood in color. Applicant considers the color versions of the drawings (which are included in priority U.S. Provisional Application No. 63/740,301, filed on Dec. 30, 2024, as well as Cornejo & Linz, ACS Sens. 10:204-212, 2025) as part of the original submission and reserve the right to present color images of the drawings in later proceedings.

FIG. 1. Cartoon illustrating particle motion tracking in thermal gel electrophoresis. (Left panel) Tracking beads (large circles) and analyte molecules (small circles, two different greys) are cast in thermal gel and then stochastically loaded throughout a microchannel. (Middle panel) Applying voltage causes soluble analyte molecules to enrich through the gel while migrating left-to-right, while large insoluble beads remain entrapped in the gel. Baseline particle motion is altered as analytes collide with the beads and/or deform the gel, which produces a quantitative signal in TGE-PMT (Right panel).

FIG. 2. Cartoon illustrating the wave-like displacement being exploited in motion tracking in thermal gel electrophoresis. Though exemplified with “sugar” as the analyte molecules, other molecules are demonstrated herein.

FIGS. 3A-3C. (FIG. 3A) PDMS containing fluorescence beads is spotted next to each feature on the master wafer near the detection point (20 mm). Fluorescence images of a device containing reference beads before (FIG. 3B) and after (FIG. 3C) loading the channel with sample gel. The same region of the same device is shown in both images. Horizontal lines indicate the channel walls.

FIG. 4. Thermal gel containing both fluorescently labeled carboxymethyl dextran and fluorescent nanoparticles was loaded into single-channel microfluidic devices. Images acquired at 470 nm (left) and 560 nm (middle) excite the FITC-sugar and the tracking beads, respectively. Both images are of the same region of the same device (just at different wavelengths) with horizontal lines added to indicate the walls of the microchannel. Data processing tracked the paths of individual beads (lines) throughout the separation (right).

FIG. 5. Electropherograms from the analysis of 500 nM FITC-CM-dextran in thermal gels containing 20-35% (w/v) Pluronic F127 at 30° C. Mean square displacement of beads is plotted versus time (n=6 beads per trace). The inset shows the same data on a zoomed-out scale to illustrate the uncontrolled migration of beads in lower gel concentrations.

FIGS. 6A-6D. (FIG. 6A) Mean squared displacement electropherograms from the analysis of 500 nM FITC-CM-dextran. Temperature was evaluated from 20-50° C. in 30% F127 (n=6 beads per trace). Data at 20° C. was omitted from the plot because beads did not remain in the field of view. The same data sets were also processed to only consider the (FIG. 6B) x-axis squared displacement and (FIG. 6C) y-axis squared displacement. Because analytes migrate along the x-axis in the direction of the electric field, only considering x-axis displacement more selectively measured the analyte peak. (FIG. 6D) Squared step size in the x-axis produced a strong response to the passing sugar band at each temperature.

FIGS. 7A-7D. Image exposure time was evaluated from 10-200 ms in the analysis of 500 nM FITC-CM-dextran. Electropherograms show (FIG. 7A) the x-axis position or (FIG. 7B) x-axis squared step size (n=6 beads per trace). Data from 10 ms exposures were omitted from the plots due to unreliable particle tracking. (FIG. 7C) Direct fluorescence measurement of the labeled sugar using 150 ms exposures. (FIG. 7D) Overlay of 150-ms traces to compare the responses of bead x-axis squared step size (“squared”), bead x-axis position (“position”), and direct fluorescence of the sugar (“DF”). Peaks aligned for clarity.

FIGS. 8A-8B. X-axis position (FIG. 8A) and x-axis squared step size (FIG. 8B) from different numbers of beads were averaged from a single device in the analysis of 500 nM FITC-carboxymethyl dextran.

FIG. 9. Signal from different numbers of beads were averaged from a single device in the analysis of 500 nM FITC-CM-dextran. Including more beads in x-axis position and x-axis squared step size data sets increased signal-to-noise until a plateau was reached.

FIGS. 10A-10B. (FIG. 10A) Image of a device with reference beads outside the channel and analysis beads inside the channel. Horizontal lines indicate channel walls. (FIG. 10B) Tracking beads from a single run of 500 nM FITC-CM-dextran were measured without (“Raw Data”) and with (“Corrected”) reference beads to correct for device drift. Data was less susceptible to baseline fluctuations and noise spikes arising from device movement when reference beads were incorporated.

FIGS. 11A-11B. Calibration curves for unlabeled carboxymethyl dextran (20 kDa) analyzed using (FIG. 11A) x-axis position TGE-PMT and (FIG. 11B) x-axis step size TGE-PMT. Detection limits are 0.63 nM and 0.52 nM, respectively.

FIG. 12. Calibration curve for unlabeled carboxymethyl dextran analyzed using UV-Vis absorbance.

FIG. 13. A sample of 5 mM 4 kDa and 40 kDa FITC-carboxymethyl dextran (two different polysaccharides) was analyzed by TGE with direct fluorescence (**) or step size PMT (black) detection. Peaks 1, 2, and 3 correspond to hydrolyzed FITC, 4 kDa FITC-CM-dextran, and 40 kDa FITC-CM-dextran, respectively.

FIGS. 14A-14B. A series of 500 nM FITC-carboxymethyl dextran samples of different molecular weights were analyzed by TGE-PMT. Peak areas were measured using both (FIG. 14A) position and (FIG. 14B) step size data. Higher molecular weight sugars yielded a greater response, indicating that analyte molecular weight affects sensitivity, not strictly analyte concentration.

FIG. 15. Fluorescein was measured either by its direct fluorescence (DF) or using label-free particle motion tracking of reporter beads (PMT).

FIGS. 16A-16B. TGE-PMT was employed to analyze native insulin (FIG. 16A), which produced one clear peak. The inset shows a corresponding negative control sample. A sample of insulin was then reduced, to break and potentially scramble disulfide bonds. Three peaks were observed (FIG. 16B). The inset shows a magnified region around the first two peaks.

### DETAILED DESCRIPTION

Aspects of the current disclosure are now described with additional details and options as follows: (I) Exemplary Definitions; (II) Label-Free Detection; (III) Particle Motion Tracking for Detection in Biomolecule Separation; (IV) Exemplary Systems for Concentration and/or Separation of Analytes; (V) Representative Device Configurations; (VI) Exemplary Electrolyte Solutions; (VII) Analytes for Analysis; (VIII) Loading of Representative Microfluidic Devices; (IX) Devices in Operation; (X) System Readout/Detection; (XI) Automated Operation using a Computer System; (XII) Kits; (XIII) Exemplary Embodiments; (XV) Experimental Examples; and (XV) Closing Paragraphs. These headings do not limit the interpretation of the disclosure and are provided for organizational purposes only.

### (I) Exemplary Definitions

Definitions and explanations used in the present disclosure are meant and intended to be controlling in any future construction when interpreting the scope of claims to avoid unnecessary vagueness or ambiguousness especially when there are more than one meanings for a certain term in different contexts.

“Analyte” refers generally to a substance (molecule, e.g., biomolecule) that is being (or to be) analyzed using a procedure or test, for instance to be identified and/or measured.

“Biomolecules” refer to molecules naturally present in organisms that are involved in one or more typically biological processes, such as cell division, morphogenesis, or development; as well as biologically active molecules (e.g., pharmaceutical drugs) that can be introduced into an organism or biological system to impact one or more biological process(s), including for the treatment of disease.

“Channel” refers to a contained space in a microfluidic device used to confine samples.

“Configured to” refers to things put together in a particular form or configuration, for instance to accomplish an intended purpose.

“Electrolyte solution” refers to a liquid or gel that contains ions, strong acids, weak acids, strong bases, and/or weak bases. The ionic compounds in an electrolyte solution are referred to as “electrolytes”. In embodiments of the current disclosure, a “Leading electrolyte solution” contains faster migrating ions (Leading electrolyte(s); LEs) than any in the sample with the same charge; while a “Trailing electrolyte solution” contains slower migrating ions (Trailing electrolyte(s); TEs) than any in the sample with the same charge. In methods and systems described in this disclosure, both cationic LEs and anionic LEs are used. In some examples, LE implies that the LE with the charge as that of the analyte (i.e. anionic). However, in some examples, LEs of both charges are needed. The same electrolytes can be used for analysis of either anionic or cationic analytes; however, the placement in reservoirs is reversed for analysis of cationic analytes compared to anionic analytes.

Also provided are electrolyte solutions that contain two or more different electrolytes, for instance for use in the herein-described zonal analysis embodiments. In such embodiments, two or more different electrolytes (each having a different characteristic electrophoretic mobility) are included in at least the anodic reservoir solution, or the cathodic reservoir solution, or both.

“Electroosmotic flow” (EOF) refers to the motion of liquid induced by an applied potential across a porous material, capillary tube, membrane, microchannel, or any other fluid conduit.

“Gel electrophoresis” refers to a process using gel (optionally, thermally-responsive gel) to conduct electrophoresis.

“Heating” refers to a process or operation for increasing the temperature of an item or an environment to be at, below, or above ambient room temperature.

“Injectionless” refers to an aspect of a process in which a sample is loaded (e.g., into a microfluidic device) without requiring injection of the sample (e.g., into an injection port).

“Microfluidics” refers to the behavior, precise control, and manipulation of fluids that are geometrically constrained to a small scale at which surface forces dominate volumetric forces. It is a multidisciplinary field that involves engineering, physics, chemistry, biochemistry, nanotechnology, and biotechnology.

“Mixed analyte” or “mixture of analytes” refers to a composition that that includes more than one analyte species, such as different species of carbohydrates, different species of nucleic acids (e.g., miRNAs), or different species of proteins or peptides. Optionally, a mixture on analytes may include different types (classes, categories) of analytes-such as both nucleic acids and proteins/peptides, or proteins/peptides and carbohydrates, and so forth. However, in embodiments described herein the mixed analyte composition only contains (or substantially only contains) one type/class/category of analyte. Alternative terms including “heterogenous analyte” and “heterogenous preparation of analytes”, for instance.

“Resolve” as used herein is a term that refers to separating an analyte mixture into distinct bands or peaks.

“Sieving gel” refers to the gel that functions such that shorter molecules move faster and migrate farther than longer ones because shorter molecules migrate more easily through the gel or through the higher viscosity gel solution.

“Thermal gel” refers to a thermally reversible compound. For example, the thermal gel may be in liquid-phase at a low temperature (e.g., 10° C.) and in solid-phase at a relatively higher temperature (e.g. 30° C.). As another example, the thermal gel may be in liquid-phase at high temperature and solid at cold temperature.

“Thermally responsive” refers to a characteristic of a substance (such as a gel) that undergoes changes in response to external temperature.

### (II) Label-Free Detection

Fluorescence provides high sensitivity and is amenable to miniaturization (Oborny et al., Analytical Sciences 32 (1): 35-40, 2016. DOI: 10.2116/analsci.32.35), making it a powerful detection scheme in analytical platforms. It has been demonstrated that fluorescence detection coupled to thermal gel electrophoresis (TGE) provides good limits of detection for diverse classes of biomolecules (see, e.g., US Patent Application Publication No. US 2023-0314370 A1; Cornejo & Linz, Analytical Chemistry, 19 (14): 5674-5681, 2022). This approach, however, requires labeling analytes, for instance with fluorescent reporters. Development of a label-free detection scheme would circumvent the need for labeling entirely. Described herein are methods to reduce the overall time and cost of the analysis by eliminating fluorescence labeling requirements. Label-free detection schemes can be integrated into analytical systems to streamline characterizations of biomedical samples.

Brownian motion describes the random movement of particles suspended in a liquid. The biosensor community has utilized this motion by conjugating capture reagents onto particles, and then introducing them into a biological sample (Colbert et al., Malaria J., 20 (1): 380, 2021. DOI: 10.1186/s12936-021-03894-w; Visser et al., ACS Nano, 10 (3): 3093-3101, 2016. DOI: 10.1021/acsnano.5b07021). Particle motion changes upon analyte binding, so by monitoring the change in particle movement, analyte can be measured. Although this approach is considered “label free”, it still requires analyte capture (e.g. protein-antibody binding) for detection. Methods described herein harness random motion to develop a truly label-free detection scheme.

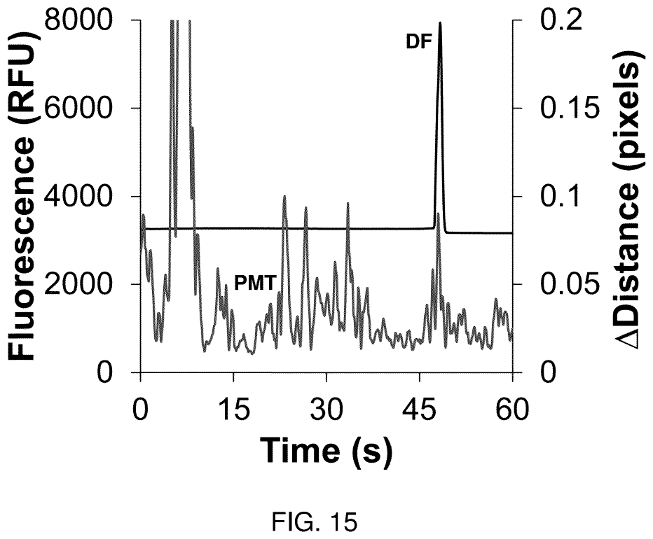

Although Brownian/random motion generally describes particles in a liquid, described herein is a system that harnesses such motion of particles entrapped in a high-viscosity thermal gel. Initial studies were conducted where fluorescent beads (200 nm) were cast into thermal gel along with a model analyte, fluorescein (500 nM), and analyzed by TGE. A control analysis was first conducted to measure the direct fluorescence of fluorescein to determine its migration time (FIG. 15, DF). This analysis also confirmed that beads did not migrate in the electric field because they were trapped in the solidified thermal gel. A second run was then conducted that tracked random motion of beads embedded in the gel at the detection point (FIG. 15, PM). A change in random motion was observed as analytes passed, including a peak with a similar migration time as the fluorescence control run. These data demonstrate that random motion of beads detected species regardless of whether they were fluorescent. This strategy enables a universal label-free detector systems. Interestingly, other peaks also appeared in the analysis that were not present in the control run. The other peaks were attributed to electrolyte bands that migrated during TGE.

In the context of this document, “label-free” refers to there not being a label attached to the biomolecule(s) being analyzed; as exemplified, label(s) may be included in other components in the analysis system.

### (III) Particle Motion Tracking for Detection in Biomolecule Separation

The current disclosure provides an improved method of analyte detection and/or quantification in gel electrophoresis, the improvement including: including tracking particles within the gel; and indirectly detecting a wavefront (band) of analyte migrating within the gel, which migrating is caused at least in part by analyte interaction with an applied electric field, the indirect detecting including detecting movement of the tracking particles in response to collisions with analyte molecules in the wavefront (band) and/or deformations of the gel. In examples of this method, the tracking particles are incapable of interacting with the analyte by binding, adsorption, or chemical reaction. Instead, the tracking particles interact through collision, such as elastic or partially elastic collision.

In example of the method embodiments herein, the tracking particles have an average diameter less than 10 microns. For instance, in embodiments the tracking particles have an average diameter selected from: ≤8 microns, ≤5 microns, ≤2 microns, ≤1 micron, ≤900 nanometers, ≤800 nanometers, ≤750 nanometers, ≤700 nanometers, ≤600 nanometers, ≤500 nanometers, ≤400 nanometers, ≤300 nanometers, ≤200 nanometers, ≤100 nanometers, ≤80 nanometers, ≤75 nanometers, ≤50 nanometers, ≤40 nanometers, 100 nanometers to 2 microns, 100 nanometers to 1 micron, 100-800 nanometers, 100-600 nanometers, 100-500 nanometers, 100-400 nanometers, 100-300 nanometers, 100-250 nanometers, 150-300 nanometers, 150-250 nanometers, 200-300 nanometers, 50-300 nanometers, 50-250 nanometers, 50-300 nanometers, 50-250 nanometers, 15-300 nanometers, 15-250 nanometers, 15-300 nanometers, 15-250 nanometers, 15 nanometers, 20 nanometers, 25 nanometers, 40 nanometers, 50 nanometers, 75 nanometers, 100 nanometers, 150 nanometers, 200 nanometers, 250 nanometers, 300 nanometers, 350 nanometers, 400 nanometers, 500 nanometers, 600 nanometers, 750 nanometers, 800 nanometers, 1 micron, or 2 microns. Specifically contemplated are embodiments of methods that employ at least two different defined-size tracking particle populations within the gel. Optionally, the average diameter of the two or more populations do not overlap, and optionally they are populations readily distinguishable using detection methods as provided herein.

In any of the provided method embodiments, detecting movement of the tracking particles can include determining displacement in an X dimension of at least one tracking particle from a time zero position, where the “x dimension” is parallel to the direction of migration of the analyte wavefront. Optionally, tracking particle displacement is calculated based on step size analysis (from one image frame to the next).

Any of the provided method embodiments can include determining the displacement of at least one, least ten, at least 100, at least 1000, at least 10,000, or more than 10,000 tracking particles.

Though exemplified using one or two defined analyte(s), it is contemplated that the provide method embodiments will also be useful for the analysis of mixed analyte samples. Therefore, the method embodiments include examples that involve indirectly detecting migration of more than one analyte, each analyte having a wavefront (band) of migration distinguishable at least in part from the wavefront (band) of other analyte(s) migrating in the gel.

Another embodiment is a method of label-free injectionless gel electrophoresis, the method including: providing a microfluidic device having a channel, the channel having a first end and a second end, the microfluidic device having a first reservoir coupled to the first end of the channel and a second reservoir coupled to the second end of the channel; loading into the channel of the microfluidic device a substantially homogenous mixture including: a mixed analyte sample; a plurality of tracking particles (for instance, nanoparticles, microparticles, or quantum dots); and a gel solution; providing a first reservoir solution in the first reservoir; providing a second reservoir solution in the second reservoir; and applying an electric field across the microfluidic device.

In any of the provided method embodiments, the tracking particles can include one or more of a polymer, a metal (such as gold or silver), silica or glass, a magnetic material, or a lipid. By way of example, tracking particles that include a polymer can include a dextran, a polystyrene, an acrylate, a poly(ethylene glycol) (PEG), a peptide, or a nucleic acid.

Also contemplated are tracking particles that are fluorescent or otherwise detectable particles. In other embodiments, the tracking particles include a detectable label, such as a dye, fluorophore, or other art-recognized label moiety.

With regard to operation of provided methods, the first reservoir solution includes a first electrolyte (at least one electrolyte) and the second reservoir solution includes a second electrolyte (at least one electrolyte). Optionally, the microfluidic device further includes a first electrode arranged in the first reservoir and a second electrode arranged in the second reservoir. By way of example, provided methods include anionic analytes migrating from the first reservoir to the second reservoir, and: the first electrode is a cathodic electrode; the first reservoir solution is a cathodic reservoir solution; the first reservoir is a cathodic reservoir; the second electrode is an anodic electrode; the second reservoir solution is an anodic reservoir solution; and the second reservoir is an anodic reservoir. In some example embodiments, the method includes cationic analytes migrating from the first reservoir to the second reservoir, and: the first electrode is an anodic electrode; the first reservoir solution is an anodic reservoir solution; the first reservoir is an anodic reservoir; the second electrode is a cathodic electrode; the second reservoir solution is a cathodic reservoir solution; and the second reservoir is a cathodic reservoir.

It is contemplated the methods can be used to analyze mixed analytes that include biomolecules. For instance, the mixed analyte sample will in various embodiments include at least one of nucleic acids, carbohydrates, peptides, or proteins. Methods that analyze mixtures of two or more of these types of these biomolecules are also provided.

Method embodiments also can further include solidifying the gel solution. By way of example, the gel is a sieving gel for resolving analytes in the sample. Optionally, the gel is thermally responsive. In examples, the gel employed in any of the described methods includes one or more of: agarose, polyacrylamide, a polymer sieving matrix, a capillary gel electrophoresis matrix, or a thermal gel polymer. Specifically contemplated thermal gel polymers include one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

Optionally, a buffer can be included in the gel solution and/or the mixed analyte sample.

Methods also provide detecting separation of analytes of the mixed analyte sample in the channel.

In any of the provided methods, optionally reference particles are embedded into or attached on at least a portion of the microfluidic device, for instance adjacent to the channel. The placement of reference particles is selected to allow imaging/detection of the reference particles concurrently with imaging/detection of tracking particles, such that movement of reference particles can be used in order to control for drift or device movement.

In any of the provided methods, the method can include indirectly detecting a wavefront of analyte migrating within the gel, which migrating results at least in part from analyte interaction with an electric field, by detecting movement of tracking particles in response to collisions of analyte molecules in the wavefront with the tracking particles and/or deformations of the gel. By way of example, the migrating (of analyte(s)) results at least in part from analyte interaction with an applied electric current and/or an applied electric voltage.

Also provided are microfluidic devices that are useful in methods provided herein. Provided microfluidic devices, in embodiments, include a channel, configured to accommodate a mixed analyte sample (e.g., containing at least one of nucleic acids, carbohydrates, peptides, or proteins) mixed with a gel solution, the channel having a first end and a second end; embedded within (or otherwise attached to) at least a portion of a wall of the channel, reference particles; a first reservoir coupled to the first end of the channel, the first reservoir being configured to accommodate a first reservoir solution; a second reservoir coupled to the second of the channel, the second reservoir being configured to accommodate a second reservoir solution; a first electrode arranged in the first reservoir; and a second electrode arranged in the second reservoir; wherein the first electrode and the second electrode are configured to apply an electric field across the microfluidic device.

Examples of such devices are configured for anionic analytes to migrate from the first reservoir to the second reservoir, and: the first electrode is a cathodic electrode; the first reservoir solution is a cathodic reservoir solution; the first reservoir is a cathodic reservoir; the second electrode is an anodic electrode; the second reservoir solution is an anodic reservoir solution; and the second reservoir is an anodic reservoir.

Additional examples of such devices are configured for cationic analytes to migrate from the first reservoir to the second reservoir, and: the first electrode is an anodic electrode; the first reservoir solution is an anodic reservoir solution; the first reservoir is an anodic reservoir; the second electrode is a cathodic electrode; the second reservoir solution is a cathodic reservoir solution; and the second reservoir is a cathodic reservoir.

In any of the provided device embodiments, the gel may be a sieving gel for resolving the sample. Optionally, the gel is thermally responsive. In examples, the gel employed in any of the described methods includes one or more of: agarose, polyacrylamide, a polymer sieving matrix, a capillary gel electrophoresis matrix, or a thermal gel polymer. Specifically contemplated thermal gel polymers include one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

In embodiments of the provided devices, the reference particles are configured as device movement/drift control reference markers.

Also provided are methods of assaying analyte migration in a liquid contained in a device (which liquid is optionally contained at least in part within a solidified gel), the method including: contacting the migrating analyte with a plurality of tracking particles of average diameter less than 10 microns, the particles being incapable of interacting with the analyte by binding, adsorption, or chemical reaction; observing motion of some or all of the tracking particles by at least one optical, fluorescence, or other electromagnetic measurement technique; and using differing motion of the tracking particles to infer presence and/or concentration of the migrating analyte.

In examples of such embodiments, the observing includes one or more of observing fluorescence, fluorescence lifetime, resonance energy transfer, phosphorescence, reflection, polarization, scattering, absorbance, chemiluminescence, or magnetic properties of some or all of the tracking particles. Optionally, the methods further including detection of electromagnetic emission (such as from detectable or labeled tracking particles and/or reference particles) at more than one wavelength.

The provided methods may further including particle tracking, single-particle tracking or tethered-particle motion tracking.

In embodiments of the methods, the analyte(s) migrating in the liquid includes electrophoretic, dielectrophoretic, isotachophoretic, or sedimentation motion. In specific examples, the analyte(s) migrating in the liquid includes electrophoresis, including through a gel.

In examples, the gel is a sieving gel for resolving migrating analytes. By way of example, the gel is thermally responsive. Specifically contemplated are embodiments in which the gel includes one or more of: agarose, polyacrylamide, a polymer sieving matrix, a capillary gel electrophoresis matrix, or a thermal gel polymer. Where the gel includes a thermal gel polymer, the polymer may include one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

The provided methods can further include observing tracking particles by camera, digital camera, PMT, scanner, microscope, telescope, detector array, time-gated, chopped, frequency-modulated, wavelength-filtered, polarization-sensitive, Raman, Surface-enhanced Raman, high numerical aperture, color-sensitive, lifetime, FRET, FRAP, intensified, phosphorescence, resistivity, ellipsometer, or high-density CCD observation, in flow, on a surface, or in suspension.

In examples of the methods, the tracking particles include nanoparticles, microparticles, or quantum dots—or mixtures of two or more thereof. Specifically contemplated are instances where two (or more) different size of tracking particles are used in the same method.

Tracking particles useful in provided methods can include one or more of a polymer, a metal (such as gold or silver), silica or glass, a magnetic material, or a lipid, where the tracking particles include a polymer, they can include a dextran, a polystyrene, an acrylate, a poly(ethylene glycol), a peptide, or a nucleic acid. Mixtures are also contemplated.

Optionally, the method involves controlling and/or monitoring a temperature of one or more elements of a system including the device used in the method.

Also provided are methods that further include assaying more than one migrating analyte, which plurality of migrating analytes are separated by the migration, and the method includes using differing motion of the tracking particles to infer presence and/or concentration of the more than one migrating analyte.

Also provided are methods that further include carrying out the analyte migrating assay in a device in including reference particles substantially equivalent in size and detection capability to the tracking particles with which the analyte(s) is contacted, wherein the method further includes controlling for device movement when calculating the differing motion of the tracking particles by detecting movement of one or more reference particles.

Yet another embodiment is an improved method of thermal gel electrophoresis (TGE), the improvement including: employing particle motion tracking (PMT) to detect and/or quantify label-free analyte(s) being subject to the TGE.

Also provided are label-free analyte separation strategies based on gel electrophoresis, and on particle motion tracking, substantially as disclosed herein.

Another embodiment is a method for separating and detecting and optionally quantifying two or more biomolecule species in a mixed sample, the method including thermal gel electrophoresis and particle motion tracking substantially as described herein.

Another example embodiment is a method of improving analyte detection in a gel electrophoresis device, including embedding or otherwise attaching reference particles in/on the gel electrophoresis device. By way of example, the embedded/attached reference particles in such methods are used to correct for device drift and/or to improve signal quality of movement of tracking particles used in the method.

(IV) Exemplary Systems for Concentration and/or Separation of Analytes

This section provides a general overview of exemplary injectionless systems for concentration and/or separation of analytes; this system is explained in additional detail in Application Publication No. US 2023-0314370 A1. Various aspects of the system will be discussed, such as the structure and elements of the system, the channel format, the gel, the electrolytes, application of current, source of power, asymmetrical electrical field, types of analytes for analysis, and the like. The system may selectively quantify analytes in a low-complexity analysis.

A microfluidic device as described herein includes a channel, a first reservoir (referred to as a cathodic reservoir or an anodic reservoir, in various embodiments), a second reservoir (referred to as an anodic reservoir or a cathodic reservoir, in various embodiments), a first electrode, and a second electrode. The channel is configured to accommodate a mixed analyte sample (that is, a sample that contains two or more different analytes) mixed with a gel solution. The channel has a first end and a second end. The first reservoir is coupled to the first end of the channel. The first reservoir is configured to accommodate a solution, e.g., a first reservoir solution. The second reservoir is coupled to the second end of the channel. The second reservoir is configured to accommodate a solution, e.g., a second reservoir solution. The first electrode is arranged in the first reservoir. The second electrode is arranged in the second reservoir. The first electrode and the second electrode, in some embodiments, are configured to apply an asymmetric electric field across the microfluidic device. Alternatively, the first electrode and the second electrode are configured to apply a symmetric electric field across the microfluidic device.

In example embodiments, the direction of the migration of analytes is from the first reservoir to the second reservoir. Where the analytes being concentrated and/or separated are anionic analytes, the migration of analytes is from the first reservoir (which is a cathodic reservoir) to the second reservoir (which is an anodic reservoir). Where the analytes being concentrated and/or separated are cationic analytes, the migration of analytes is from the first reservoir (which is an anodic reservoir) to the second reservoir (which is a cathodic reservoir). Additional embodiments contemplated.

### Microfluidic Device on a Microscope Slide or Other Portable Unit:

Microfluidic devices in this demonstration are molded channels in polydimethylsiloxane. Individual channels were diced from the mold and then adhered to glass slides to form enclosed channels to contain fluid. Devices can also be created from other materials including, but not limited to, cyclic olefin copolymer, cyclic olefin polymer, acrylic, acrylonitrile butadiene styrene, nylon polyamide, polycarbonate, polyethylene, polyoxymethylene, polypropylene, polystyrene, polyurethane, or glass. Devices can also be created using processes including injection molding, hot embossing, and the like.

Straight-sided (standard) channels vs. tapered channel: Microfluidic devices may have a straight-sided channel (standard channels) or a tapered channel, as described herein. The tapered channel microfluidic device was designed to further improve detection sensitivity and separation resolution. The tapered channel was created to confine analytes into bands that progressively migrated into regions of higher electric field. This novel device design significantly improved limits of detection and separation resolution compared to a standard channel microfluidic device.

Gels: As described herein, thermal gels can be used during the process of injectionless gel electrophoresis. Example thermal gels include Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA) and N-methoxyethylacrylamide (NMEA). Other types of gels, such as matrices for capillary gel electrophoresis (Miksík et al., Biomed. Chromatogr. 20:458-465, 2006), polymer sieving matrices (Chung et al., The Royal Society of Chem., 139:5635-5654, 2014), and the like may be used. The present disclosure is not limited thereto.

Electrolyte solutions (Leading and Trailing): Electrolyte solution(s) are used to provide ions that carry a current and to conduct the preconcentration and electrophoresis processes. In representative embodiments described herein, a first reservoir and a second reservoir are configured to accommodate electrolyte solutions. As described herein, the electrolyte solutions include anionic and/or cationic components. In some examples, the electrolyte solution may include a trailing electrolyte (TE) solution and a leading electrolyte (LE) solution. There are four classes of electrolytes: LE+, LE−, TE+, TE−. In some examples, both anionic LE (LE−) and anionic TE (TE−) can be added into the same reservoir. One representative example where the first reservoir is a cathodic reservoir, the cathodic reservoir solution contains 800 mM glycine (which is TE−), 5 mM tris-HCl (where tris is TE+ and Cl− is LE−), and 1 mM MgCl2 (where Mg²⁺ is LE+, and Cl− is LE−), and the anodic reservoir solution (contained in the second reservoir, which is an anodic reservoir) is composed of 200 mM ammonium acetate (where ammonium is LE+, and acetate is LE−), 5 mM tris-HCl, and 1 mM MgCl2 (for instance, if nucleic acids are being analyzed). Additional representative examples include: the cathode reservoir solution contains 400 mM glycine, 2 mM tricine, and 5 mM tris-HCl (pH 7.5) and the anode reservoir solution contains 30 mM tris-HCl (pH 8.0), 200 mM ammonium acetate (for instance, as illustrated in Example 2); and the cathode reservoir solution contains 800 mM glycine and 5 mM tris-HCl, and the anode reservoir solution contains 5 mM tris-HCl, 10 mM ammonium acetate, 30% Pluronic F127 (for instance, as illustrated in Example 3).

The same electrolytes can be used for analysis of either anionic or cationic analytes; however, the placement in reservoirs is reversed for analysis of cationic analytes compared to anionic analytes.

More generally, exemplary electrolytes include: Glycine (TE−); Tris-HCl (where Tris is TE+ and Cl is LE−); MgCl₂ (where Mg²⁺ is LE+ and Cl− is LE−); Ammonium acetate (where ammonium is LE+ and acetate is LE−); Tricine (TE−); Proline (TE−); Borate (TE−); HEPES (TE−); Bis-tris methane (TE+); Bis-tris propane (TE+); NaCN (where sodium is LE+ and CN is LE−); NaCl (where sodium is LE+ and Cl is LE−); ammonium chloride (where ammonium is LE+ and Cl is LE−); and sodium acetate (where sodium is LE+ and acetate is LE−). Additional feasible electrolytes will be readily identified by those of skill in the art. Further, one of ordinary skill can order the relative mobility of any two (or more) electrolyte species, for instance in order to use two (or more) in a multi-zonal TGE analysis as described herein.

Application of Current: Electrophoresis voltage is applied across the device via the electrodes using a high-voltage power supply. For example, the voltage applied may be ±1 kV for the standard channel device, and ±2 kV for the tapered channel device. Current could be applied across the device instead of voltage to drive the analysis. This illustrated designation of negative electrode and ground applies for analysis of anionic analytes. It will be understood by one of ordinary skill in the relevant art that the disclosure also provides configurations in which the ground is replaced with a positive electrode. Alternatively, the polarity could be reversed from positive to ground or positive to negative, which would be employed for analysis of cationic analytes. In embodiments, the second electrode is not grounded but instead is held at a potential.

Source of Power: A four-channel high voltage power supply (Advanced Energy, Ronkonkoma, NY) was used to apply an electric field across the microfluidic channel. Gel electrophoresis as used herein generally operates with simplified hardware requirements (e.g., there is no need for a second power supply nor timing actuator) to reduce cost of the system and increase ease of operation (for instance, in comparison to MCE). As an example, the power may be less than 0.2 Watt.

Asymmetrical Electrical Field: In some embodiments, the first electrode and the second electrode are configured to apply an asymmetric electric field across the microfluidic device. The optional use of an asymmetric electric field better confines analytes to reduce volume and increase concentration. In embodiments analyzing cationic analytes, offset electrodes deflect analytes to the opposite channel wall because of repulsion from the anode.

### Types of Analytes for Analysis

In implementations, the sample may include at least one of nucleic acids, carbohydrates, peptides, or proteins. As described, the processes of particle motion tracking for detection in biomolecule separation can be used to analyze various types of analytes, such as proteins, carbohydrates, peptides, or other biomolecules. Moreover, conditions (such as electrolytes, voltages, dimensions of the microfluidic device, and so on) for conducting analysis may be varied for different analytes. For example, when analyzing nucleic acids, proteins or other analytes, they will be separated in the provided systems based on their own physical and charge characteristics.

Proteins must maintain proper folding conformations and express the correct post-translational modifications (PTMs) to exhibit appropriate biological activity. However, assessing protein folding and PTMs is difficult because routine polyacrylamide gel electrophoresis (PAGE) methods lack the separation resolution necessary to identify variants of a single protein. Additionally, standard PAGE denatures proteins prior to analysis precluding determinations of folding states or PTMs. To overcome these limitations, a microfluidic gel electrophoresis platform that provides high-sensitivity, high-resolution analyses of native protein variants can be used (e.g., as described in Application Publication No. US 2023-0314370 A1). In that exemplary system, thermally reversible gel is utilized as a separation matrix while in its solid state (30° C.). This gel provides sufficient separation resolution to identify variants of proteins. To increase detection sensitivity, analyte preconcentration can be conducted in parallel with the separation. The unique temperature dependent outcomes in Application Publication No. US 2023-0314370 A1 illustrated how method performance can be tuned through a thermal dimension. Ultimately, the high detection sensitivity and separation resolution provided by gel electrophoresis enables rapid screening of native protein variants. Protein variants can be separated in an analogous method/mechanism as the miRNAs.

The same power supply can be used as in analysis of any chosen analyte or mixture of analytes. Temperature of the stage can be controlled, for instance using a suitable equipment (e.g., Peltier (TEC1-12730)) and thermoelectric controller (Wavelength Electronics, Bozeman, MT). Real-time temperature feedback can be provided, for instance using a resistance temperature detector (Omega Engineering, Norwalk, CT) affixed to the Peltier. Separation voltage and stage temperature can be controlled via computer. Images can be acquired, stored, and analyzed using appropriate equipment. For instance, images can be acquired at 2.4× magnification with 150 ms exposure times at discrete distances along the separation channel. Hardware can be controlled using MicroManager. Images can be processed using FIJI and field-flattened to correct for nonuniform illumination across the channel. Separation metrics can be determined, for instance, using Chromophoreasy software (Vaz et al., J. Brazil Chem. Soc., 27:1899-1911, 2016).

### (V) Representative Device Configurations

This section provides details of electrophoresis device configuration including the structure and elements of exemplary microfluidic separation devices and how they work to perform their function.

Overall structure: a microfluidic device, including a channel, a first reservoir, a second reservoir, a first electrode, and a second electrode. The channel is configured to accommodate a mixed analyte sample (that is, a sample that contains two or more different analytes) mixed with a gel solution. The channel has a first end and a second end. The first reservoir is coupled to the first end of the channel. The first reservoir is configured to accommodate a first reservoir solution. The second reservoir is coupled to the second end of the channel. The second reservoir is configured to accommodate a second reservoir solution. The first electrode is arranged in the first reservoir. The second electrode is arranged in the second reservoir. The first electrode and the second electrode, in some embodiments, are configured to apply an asymmetric electric field across the microfluidic device.

Channel formats: different channel formats can be used here, such as straight-sided (standard) channel, tapered channel, serpentine channel, or any combination thereof. This disclosure is not limited thereto, and other shapes or formats of the channel can be used as long as it can perform the injectionless gel electrophoresis process.

Reservoir placement and formats: the reservoirs are placed at two ends of the channel of the microfluidic device. As an example, the first reservoir (cathodic reservoir) and the second reservoir (anodic reservoir) are configured to accommodate the electrolyte solutions. In some examples, the electrolyte solution may include the cathodic reservoir solution and the anodic reservoir solution. The positions of the reservoirs relative to the channel affect the analysis, especially in the application of an asymmetric electric field.

Electrode placement and format: The first electrode is arranged in the first reservoir. The second electrode is arranged in the second reservoir. In some embodiments, the first and second electrodes are configured to apply an asymmetric electric field using offset electrode positions. As described herein, the offset electrode positions may deflect analytes to the opposite side of the channel because of the asymmetric electric field and Coulombic repulsion of the anionic nucleic acids from the cathode. In embodiments analyzing cationic analytes, offset electrodes deflect analytes to the opposite channel wall because of repulsion from the anode. Alternatively, the first electrode and the second electrode are configured to apply a symmetric electric field across the microfluidic device.

In some embodiments, the method involves anionic analytes migrating from the first reservoir to the second reservoir, and the first electrode is a cathodic electrode; the first reservoir solution is a cathodic reservoir solution; the first reservoir is a cathodic reservoir; the second electrode is an anodic electrode; the second reservoir solution is an anodic reservoir solution; and the second reservoir is an anodic reservoir.

In some embodiments, the method comprises cationic analytes migrating from the first reservoir to the second reservoir, and the first electrode is an anodic electrode; the first reservoir solution is an anodic reservoir solution; the first reservoir is an anodic reservoir; the second electrode is a cathodic electrode; the second reservoir solution is a cathodic reservoir solution; and the second reservoir is a cathodic reservoir.

This illustrated designation of negative electrode and ground applies for analysis of anionic analytes. It will be understood by one of ordinary skill in the relevant art that the disclosure also provides configurations in which the ground is replaced with a positive electrode. Alternatively, the polarity could be reversed from positive to ground or positive to negative, which would be employed for analysis of cationic analytes. In embodiments, the second electrode is not grounded but instead is held at a potential.

Power supply: different types of power supply can be used here. As an example, a four-channel high voltage power supply (Advanced Energy, Ronkonkoma, NY) was used to apply an electric field across the microfluidic channel. This disclosure is not limited thereto, and other types of power supply can be used as long as it can perform the injectionless gel electrophoresis process.

Additionally, those having ordinary skills in the art readily recognize that the techniques described above can be utilized in a variety of devices, environments, and situations. Although the subject matter has been described in language specific to structural features or methodological acts, it is to be understood that the subject matter defined in the appended claims is not necessarily limited to the specific features or acts described. Rather, the specific features and acts are disclosed as exemplary forms of implementing the claims.

Those of ordinary skill in the art will recognize in light of the present disclosure that many changes can be made to the specific embodiments disclosed herein and still obtain a like or similar result without departing from the spirit and scope of the disclosure.

### (VI) Exemplary Electrolyte Solutions

Electrolyte solutions are used to provide ions that carry a current and to conduct the electrophoresis process. These solutions have abundant ions in them, which is necessary for the passage of electricity through them. In devices useful in embodiments described herein, the first reservoir and the second reservoir are configured to accommodate the electrolyte solutions. In some examples, the electrolyte solution may include the cathodic reservoir solution and anodic reservoir solution. As described herein, the placement of a cathodic reservoir solution or an anodic reservoir solution in the first or second reservoir is influenced by the type(s) of analytes being concentrated and/or separated. For instance, where analytes being concentrated and/or separated are anionic analytes, the migration of analytes is from the first reservoir (which is then a cathodic reservoir) to the second reservoir (which is then an anodic reservoir). Alternatively, where the analytes being concentrated and/or separated are cationic analytes, the migration of analytes is from the first reservoir (which is then an anodic reservoir) to the second reservoir (which is then a cathodic reservoir).

The same electrolytes can be used for analysis of either anionic or cationic analytes; however, their placement in reservoirs is reversed for analysis of cationic analytes compared to anionic analytes. Exemplary electrolytes include: Glycine (TE−); Tris-HCl (where Tris is TE+ and Cl is LE−); MgCl₂ (where Mg²⁺ is LE+ and Cl− is LE−); Ammonium acetate (where ammonium is LE+ and acetate is LE−); Tricine (TE−); Proline (TE−); Borate (TE−); HEPES (TE−); Bis-tris methane (TE+); Bis-tris propane (TE+); NaCN (where sodium is LE+ and CN is LE−); NaCl (where sodium is LE+ and Cl is LE−); ammonium chloride (where ammonium is LE+ and Cl is LE−); and sodium acetate (where sodium is LE+ and acetate is LE−). Additional feasible electrolytes will be readily identified by those of skill in the art. Further, one of ordinary skill can order the relative mobility of any two (or more) electrolyte species, for instance in order to use two (or more) in a multi-zonal TGE analysis as described herein.

Though example specific electrolytes, and combinations of electrolytes, are described, one of ordinary skill in electrophoresis and related systems will understand that additional electrolytes can be used in the described systems and methods, and will understand how to select an electrolyte or combination of different electrolyte species based on their mobility characteristics in order to facilitate analyte separation based on the teachings herein.

Concentrations: As an example, the cathodic reservoir solution is composed of 800 mM glycine, 5 mM tris-HCl, and 1 mM MgCl₂, and the anodic reservoir solution is composed of 200 mM ammonium acetate, 5 mM tris-HCl, and 1 mM MgCl₂.

Variation based on target analyte: In implementation, the selection and concentrations of electrolyte(s) may differ based on target analyte(s) to be separated/analyzed. For example, miRNA analyses use 800 mM glycine, and protein analyses use 100 mM glycine. Embodiments of miRNA analyses use 200 mM ammonium acetate, and embodiments of protein analyses use 10 mM ammonium acetate. Additional examples are provided herein, and can be selected by those of skill in the art.

Placement in device: LE and TE compositions may be optimized to maximize analyte enrichment, for instance until the band reaches approximately half-way to the detection point followed by an automatic initiation of the separation. The closest analogy of the sought behavior in the literature are reports of bidirectional ITP (Bahga et al., Anal. Chem., 83 (16): 6154-6162, 2011). Although the arrangement of electrolytes in the herein described injectionless gel electrophoresis system is inconsistent with bidirectional ITP, similar components are employed.

Additional options: Any anionic electrolytes that meet the following condition can be used:

$${μ}_{TE -} < {μ}_{Analyte} < {μ}_{LE -}$$

where μ is mobility. This essentially means that the analyte would have an intermediate mobility between the LE and TE. Other examples of TE− that can be used include tricine, borate, HEPES, proline, and the like. Other examples of LE− that can be used include acetate and the like.

A high mobility electrolyte (LE+, e.g. ammonium) and a low mobility electrolyte (TE+, e.g. tris) are used to initiate the separation.

Variations in order to Provide Multiple Zones: Representative electrophoretic systems can be operated in a “zonal” mode, or a multi-zonal mode (given that typical operation may be considered a “single zone” mode) that enables separation of more analytes or analytes with otherwise “too crowded” migration characteristics. In the multi-zonal mode, the electrolyte solution used in the anodic reservoir, or in the cathodic reservoir, or both, includes more than one electrolyte with different mobility characteristics. The plurality of electrolytes is selected to allow formation of zones of separation through the analysis process; these zones are formed where the following conditions are met for the mobility of each member of the system: Analyte₁>TE−₁>Analyte₂>TE−₂ (for an exemplary two-zone anionic electrolyte system).

For instance, separation resolution increases in analyte separation (for instance, samples in which miRNA and/or proteins are being analyzed) when a second anionic TE is added into the cathodic reservoir. This second TE forms an additional zone in which analytes can separate after undergoing preconcentration. This two-TE approach (which is an example of a multi-zonal electrophoretic system) increases flexibility of the analysis by enabling high separation resolution between both higher mobility analytes and lower mobility analytes in a single analysis. This approach can be extended further by incorporating more electrolytes (e.g., tricine, proline, or the like) into the analysis, which further increases the flexibility of TGE to analyze samples of even higher complexity.

Proline can serve as a low-mobility anionic TE in the analysis of large proteins. Glycine can be used along with proline to resolve proteins of moderate mobility from proteins of low mobility in separate zones. In principle, a third anionic TE of higher mobility (e.g., tricine) can be added into the cathodic reservoir solution to form a third separation zone. Using three anionic TEs is expected to enhance resolution between analytes of high mobility, moderate mobility, and low mobility using the same TGE format as in previous examples. This approach can also extend to greater numbers of electrolytes, and is not limited to one or two anionic TEs.

TGE enables analyses to be readily customized based on the analytes in a sample mixture. Multiple TEs can be combined to accentuate resolution between sets of analytes that differ in mobilities. The number of separation zones needed for analyzing a given sample may be influenced the number of different analyte species present and their relative mobility differences.

In principle, similar customization can be attained by using additional cationic electrolytes in the anodic reservoir. Distinct cationic electrolyte zones will migrate counter to the direction of the analytes, which influences the separation resolution and preconcentration efficiency. Having the flexibility to adjust the electrolyte composition in one or both reservoirs and obtain superior analytical performance further expands the utility of TGE for biomolecular analyses.

### (VII) Analytes for Analysis

Methods and systems described here may be used for preconcentrating and/or separating and/or quantitating various types of analytes.

Type of analytes: Analytes for analysis include biomolecules. In implementations, the sample may include at least one of nucleic acids, carbohydrates, peptides, or proteins. In implementations, the sample may include two or more nucleic acid species, such as two or more miRNA species. Additional details of the sample are described throughout the present disclosure.

Another way to divide types of analytes is whether they are anionic (that is, having a negative net charge in the system) or cationic (having a positive net charge in the system). This is relevant in part because the configuration of the microfluidic device may be customized for analysis based on analyte charge. Where the analytes being concentrated and/or separated are anionic analytes, the migration of analytes is from the first reservoir (which is then a cathodic reservoir) to the second reservoir (which is then an anodic reservoir). Where the analytes being concentrated and/or separated are cationic analytes, the migration of analytes is from the first reservoir (which is then an anodic reservoir) to the second reservoir (which is then a cathodic reservoir).

Heterogeneity of Analyte Mixture: In implementations, the analyte mixture may include different types of molecules. In some embodiments, the analyte mixture may be from medical samples, environmental samples, laboratory samples, samples from human patients, animal subjects, etc. In some embodiments, the analyte mixture may contain a heterogenous collection of different analytes—that differ by size, shape, isoelectric point, and so forth.

In analyzing heterogenous analyte mixtures, it may be beneficial to operate the provided systems using more than one zone of separation. Employing multiple zones enables separation of target analytes with divergent mobility in the system, or mixtures that have mobilities that are overlapping when separated without using multi-zonal separation.

In some examples, the analyte may include carbohydrates, peptides, or proteins. For the analyte that includes carbohydrates, peptides, or proteins, probes may not be needed. It is also specifically contemplated that labels are not required for the processes of particle motion tracking for detection in biomolecule separation.

### (VIII) Loading of Representative Microfluidic Devices

It is a benefit of exemplary embodiments that a microfluidic device may not require a specialized type of loading system (such as injection), and instead the analyte-containing sample is simply loaded throughout a single microfluidic channel of the microfluidic device. In such systems no sample injection is needed to begin the analysis, unlike standard analytical methods.

Immobilization composition (Thermal gel and other types of immobilizer): In some examples, a thermal gel is used that is liquid at cool temperatures. The microfluidic device is placed in a cold environment (e.g., on an ice bath, in a cold room) to keep the gel liquid. A drop of gel is placed in the first reservoir. The gel is loaded into the channel by applying vacuum at the second reservoir, applying pressure at the first reservoir, allowing capillary action to transport the gel from the reservoir into the channel, or some combination of two or more thereof. Once the channel is filled, excess gel is removed from the reservoirs. The device is then removed from the cold environment, which causes the gel to solidify. The analytes and electrolytes are now immobilized in the solid gel throughout the entirety of the channel. Cathodic and anodic reservoir solutions are then added to the first and the second reservoirs, respectively. Then, the device is ready to be operated (i.e., apply voltage, detect analytes, etc.).

As described herein, thermal gels can be used during the process of particle motion tracking for detection in biomolecule separation. Thermal gels have been previously reported to help filter miRNAs from other nucleic acids (Schoch et al., Lab Chip, 9 (15): 2145-2152, 2009; Han et al., Lab Chip, 19 (16): 2741-2749, 2019). Example thermal gels include Pluronic F-127 (aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA) and N-methoxyethylacrylamide (NMEA). The present disclosure is not limited thereto. Other gels, such as matrices for capillary gel electrophoresis (Miksík et al., Biomed. Chromatogr. 20:458-465, 2006), polymer sieving matrices (Chung et al., The Royal Society of Chem., 139:5635-5654, 2014), and the like may be used.

Mixing sample: In some embodiments, the stock sample is prepared containing mixtures of compounds (e.g., biomolecules and labels—such as detection or drag probes). Sample is directly cast into gel and non-selectively loaded throughout the entirety of a single-channel microfluidic device, which increases the user-friendliness. As an example, the sample (or sample-probe mixture) may be cast into gel at a 1:9 ratio. As an example, the final samples contained 30% (w/v) PF-127 and 1 mM MgCl₂ in 20 mM tris-HCl, with variable concentrations of nucleic acid molecules and 10 nM probes.

Buffer inclusion: Buffers in gel electrophoresis are used to provide ions that carry a current and to maintain the pH at a relatively constant value. Buffer can be included in the gel. In an example, the buffer is tris-HCl.

Solidification of sample into channel: In embodiments in which a thermal gel that is liquid at cool temperatures is used, all solutions are prepared and stored on ice prior to analysis. After loading the analyte-containing composition into the microfluidic device, the gel is then solidified in place by warming the device (e.g. 25° C.). This effectively immobilizes the sample analytes and electrolytes in place, and may provide a sieving matrix.

### (IX) Devices in Operation

Devices as described herein can operate to conduct the electrophoresis and particle motion tracking for detection in biomolecule separation, to analyze various types of analytes. The following provides representative description of various aspects of devices in operation.

Power source: Gel electrophoresis as used herein generally operates with simplified hardware requirements (e.g., there is no need for a second power supply nor timing actuator) to reduce cost of the system and increase ease of operation (for instance, in comparison to MCE). As an example, the power may be 0.2 Watt. In exemplary embodiments, a four-channel high voltage power supply (Advanced Energy, Ronkonkoma, NY) was used to apply an electric field across the microfluidic channel.

Buffer maintenance: Buffers in gel electrophoresis are used to provide ions that carry a current and to maintain the pH at a relatively constant value. Buffer can be included in the gel.

Voltage application: Electrophoresis voltage is applied across the device via the electrodes using a high-voltage power supply. For example, the voltage applied may be ±1 kV for the standard channel device, and ±2 kV for the tapered channel device. In embodiments, −1 kV was applied for instance for analysis of anionic analytes. In embodiments analyzing cationic analytes, a positive voltage (e.g., ±1 kV) is used. As an example, the power may be less than 0.2 Watt.

Timing: As described herein, TGE including when coupled with particle motion tracking for detection in biomolecule separation operates with simplified hardware requirements (e.g. no second power supply nor timing actuator) to reduce cost of the system and increase ease of operation versus MCE.

Temperatures: In examples, samples are stored at −20° C. Thermal gel stock solution is prepared by dissolving 33.3% (w/v) Pluronic F-127 in 20 mM tris-HCl at 4° C. to maintain the gel in a liquid state. TGE employs a thermally responsive polymer that changes viscosity in response to temperature (Durney et al., Anal. Chem., 85 (14): 6617-6625, 2013). Sample is cast directly into liquid-phase thermal gel (e.g. 10° C.) for facile loading into microfluidic channels (Burton et al., Anal. Methods, 11 (37): 4733-4740, 2019). The gel is then solidified by warming the device (e.g. 25° C.) to immobilize the sample, particles, and electrolytes in place and to provide a sieving matrix. Filled devices are analyzed using a microscope, such as an AZ100 epifluorescent microscope (Nikon Instruments Inc., Melville, NY). Before analysis, the devices are allowed to equilibrate e.g. at 25° C. for 2 min on a temperature-controlled stage.

In embodiments, the sample analysis is carried out at a temperature between 5° C. and 65° C., for instance at 5° C., 10° C., 15° C., 20° C., 25° C., 30° C., 35° C., 40° C., 45° C., 50° C., 55° C., 60° C., 65° C., or higher; or at any individual temperature in the range of 5° C. to 65° C. In general, a system can be said to be operated at “high” temperature when it is operated at 45° C. or higher.

### (X) System Readout/Detection

The system may have readouts and detection results for different purpose such as clinical and pharmaceutical purpose.

Camera/detector: In some embodiments, images are acquired during analysis, for instance using an ORCA Fusion sCMOS camera (Hamamatsu Corp., Bridgewater, NJ) with 150 ms exposure times. Excitation light was produced in exemplary embodiments using by a SOLA Light Engine (Lumencor, Beaverton, OR) with a Texas Red filter cube (560/630 nm) at an intensity of 5 mW/mm², though other systems may be used and the appropriate filter(s) selected based on the label(s) being detected. A movable stage (exemplified by those available from Prior Scientific, Rockland, MA) may be used to track analyte migration. μManager software or equivalent is used to control all hardware and trigger image acquisition (Edelstein et al., Curr. Protoc. Mol. Biol., 92 (1): 14.20.1-14.20.17., 2010).

Computer system: Methods and processes described herein may be implemented by a computer system. Computer-executable instructions stored on one or more computer-readable storage media, when executed by the computer system, cause the computer system to perform the recited operations. Generally, computer-executable instructions include routines, programs, objects, components, data structures, and the like that perform particular functions or implement particular abstract data types. Those having ordinary skills in the art will readily recognize that certain steps or operations illustrated in the figures above may be eliminated, combined, or performed in an alternate order. Any steps or operations may be performed serially or in parallel (unless the context requires one or the other). Furthermore, the order in which the operations are described is not intended to be construed as a limitation.

### (XI) Automated Operation Using a Computer System

The methods and devices described herein can be used for automated analysis of biomolecules, exemplified herein with carbohydrates. Applications for the herein described methods and devices include separating, detecting, and/or measuring biomarkers (more generally, analytes) for clinical diagnostics and performing quality control analyses of pharmaceutical formulations.

Further, the processes discussed herein may be implemented in hardware, software, or a combination thereof. In the context of software, the described operations represent computer-executable instructions stored on one or more computer-readable storage media that, when executed by one or more hardware processors, perform the recited operations. Generally, computer-executable instructions include routines, programs, objects, components, data structures, and the like that perform particular functions or implement particular abstract data types. Those having ordinary skills in the art will readily recognize that certain steps or operations illustrated in the figures above may be eliminated, combined, or performed in an alternate order. Any steps or operations may be performed serially or in parallel (unless the context requires one or the other). Furthermore, the order in which the operations are described is not intended to be construed as a limitation.

Embodiments may be provided as a software program or computer program product including a non-transitory computer-readable storage medium having stored thereon instructions (in compressed or uncompressed form) that may be used to program a computer (or other electronic devices) to perform processes or methods described herein. The computer-readable storage medium may be one or more of an electronic storage medium, a magnetic storage medium, an optical storage medium, a quantum storage medium, and so forth. For example, the computer-readable storage media may include, but is not limited to, hard drives, floppy diskettes, optical disks, read-only memories (ROMs), random access memories (RAMs), erasable programmable ROMs (EPROMs), electrically erasable programmable ROMs (EEPROMs), flash memory, magnetic or optical cards, solid-state memory devices, or other types of physical media suitable for storing electronic instructions. Further, embodiments may also be provided as a computer program product including a transitory machine-readable signal (in compressed or uncompressed form). Examples of machine-readable signals, whether modulated using a carrier or unmodulated, include, but are not limited to, signals that a computer system or machine hosting or running a computer program can be configured to access, including signals transferred by one or more networks. For example, the transitory machine-readable signal may include the transmission of software by the Internet.

Separate instances of these programs can be executed on or distributed across any number of separate computer systems. Thus, although certain steps have been described as being performed by certain devices, software programs, processes, or entities, this need not be the case, and a variety of alternative implementations will be understood by those having ordinary skills in the art.

### (XII) Kits

The systems and methods disclosed herein can be employed using kits. Disclosed kits include materials and reagents necessary to assay a sample obtained from a subject for diagnosis and/or detection of biomarkers for diagnosing pathologies including cancers (Cheng, Adv. Drug Deliver. Rev., 81:75-93, 2015; Ban, J. Chromatogr. A, 1315:195-199, 2013), cardiovascular diseases (Zhu & Fan, Am. J. Cardiovasc. Dis., 1:138-149, 2011; Creemers et al., Circ. Res., 110 (3): 483-495, 2012), and neurodegenerative disorders (Femminella et al., Front. Physiol., 6, 2015; Sheinerman & Umansky, Front. Cell. Neurosci., 7:150-150, 2013; Du & Pertsemlidis, J. Mol. Cell Biol., 3:176-180, 2011), quality control for pharmaceuticals, validating biological research samples, etc. In particular embodiments, the kit includes at least one of: (1) control analytes; (2) a gel or mixture of gels, such as Pluronic F-127 (aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethylene-oxide), N,N′-dimethylacrylamide (DMA) and N,N′-diethylacrylamide (DEA), N-ethoxyethylacrylamide (NEEA) and N-methoxyethylacrylamide (NMEA), matrices for capillary gel electrophoresis (Miksík et al., Biomed. Chromatogr. 20:458-465, 2006), polymer sieving matrices (Chung et al., The Royal Society of Chem., 139:5635-5654, 2014), and the like; (3) microfluidic device(s) such as straight-sided channels (standard channels) devices and tapered channel devices as described herein; (4) one or more cathodic reservoir electrolytes (such as glycine, tris-HCl, and/or MgCl₂, or other electrolytes as provided herein, for the analysis of anionic analytes), optionally in solution; (5) one or more anodic reservoir electrolytes (such as ammonium acetate, tris-HCl, and/or MgCl₂, or other electrolytes as provided herein, for the analysis of anionic analytes), optionally in solution; and/or (6) buffers, e.g., IDTE buffer (10 mM Tris, 0.1 mM ethylenediaminetetraacetic acid, pH 7.5). The same electrolytes can be used for analysis of either anionic or cationic analytes; however, the placement in reservoirs is reversed for analysis of cationic analytes. One of ordinary skill in electrophoresis and related systems will understand that additional electrolytes can be used in the described systems and methods, and will understand how to select an electrolyte or combination of different electrolyte species based on their mobility characteristics in order to facilitate analyte separation based on the teachings herein.

Components of the kits can be packaged in aqueous media or in lyophilized form. The container means of the kits can include at least one vial, test tube, flask, bottle, syringe or other container means, into which a component may be placed, and preferably, suitably aliquoted. Where there is more than one component in the kit, the kit can include a second, third or other additional container into which the additional components may be separately placed. The kits may also include a second container means for containing a sterile, pharmaceutically acceptable buffer and/or other diluent. In particular embodiments, various combinations of components may be included in a vial.

The kit may include instructions for employing the kit components as well as the use of any other reagent not included in the kit. Instructions may include variations that can be implemented.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (XIII) Exemplary Embodiments

1. An improved method of analyte detection and/or quantification in gel electrophoresis, the improvement including: including tracking particles within the gel; and indirectly detecting a wavefront (band) of analyte migrating within the gel, which migrating is caused at least in part by analyte interaction with an applied electric field, the indirect detecting including detecting movement of the tracking particles in response to collisions with analyte molecules in the wavefront (band) and/or deformations of the gel.

2. The method of embodiment 1, wherein the tracking particles are incapable of interacting with the analyte by binding, adsorption, or chemical reaction.

3. The method of embodiment 1, wherein the tracking particles have an average diameter less than 10 microns.

4. The method of embodiment 3, wherein the tracking particles have an average diameter selected from: ≤8 microns, ≤5 microns, ≤2 microns, ≤1 micron, ≤900 nanometers, ≤800 nanometers, ≤750 nanometers, ≤700 nanometers, ≤600 nanometers, ≤500 nanometers, ≤400 nanometers, ≤300 nanometers, ≤200 nanometers, ≤100 nanometers, ≤80 nanometers, ≤75 nanometers, ≤50 nanometers, ≤40 nanometers, 100 nanometers to 2 microns, 100 nanometers to 1 micron, 100-800 nanometers, 100-600 nanometers, 100-500 nanometers, 100-400 nanometers, 100-300 nanometers, 100-250 nanometers, 150-300 nanometers, 150-250 nanometers, 200-300 nanometers, 50-300 nanometers, 50-250 nanometers, 50-300 nanometers, 50-250 nanometers, 15-300 nanometers, 15-250 nanometers, 15-300 nanometers, 15-250 nanometers, 15 nanometers, 20 nanometers, 25 nanometers, 40 nanometers, 50 nanometers, 75 nanometers, 100 nanometers, 150 nanometers, 200 nanometers, 250 nanometers, 300 nanometers, 350 nanometers, 400 nanometers, 500 nanometers, 600 nanometers, 750 nanometers, 800 nanometers, 1 micron, or 2 microns.

5. The method of embodiment 1, involving including at least two different defined-size tracking particle populations within the gel.

6. The method of embodiment 1, wherein detecting movement of the tracking particles includes determining displacement in an X dimension (parallel to the direction of migration of the analyte wavefront) of at least one tracking particle from a time zero position.

7. The method of embodiment 6, wherein tracking particle displacement is calculated based on step size analysis (from one image frame to the next).

8. The method of embodiment 6, including determining the displacement of at least one, at least two, at least three, at least four, at least five, at least seven, at least ten, at least 50, at least 100, at least 1000, at least 10,000, or more than 10,000 tracking particles.

9. The method of embodiment 1, including indirectly detecting migration of more than one analyte, each analyte having a wavefront (band) of migration distinguishable at least in part from the wavefront (band) of other analyte(s) migrating in the gel.

10. A method of label-free injectionless gel electrophoresis, including: providing a microfluidic device having a channel, the channel having a first end and a second end, the microfluidic device having a first reservoir coupled to the first end of the channel and a second reservoir coupled to the second end of the channel; loading into the channel of the microfluidic device a substantially homogenous mixture including: a mixed analyte sample; a plurality of tracking particles; and a gel solution; providing a first reservoir solution in the first reservoir; providing a second reservoir solution in the second reservoir; and applying an electric field across the microfluidic device.

11. The method of embodiment 10, wherein the tracking particles include nanoparticles, microparticles, or quantum dots.

12. The method of embodiment 10, wherein the tracking particles include one or more of a polymer, a metal (such as gold or silver), silica or glass, a magnetic material, or a lipid.

13. The method of embodiment 12, wherein the tracking particles include a polymer including a dextran, a polystyrene, an acrylate, a poly(ethylene glycol), a peptide, or a nucleic acid.

14. The method of embodiment 10, wherein the tracking particles include a detectable label.

15. The method of embodiment 10, wherein the first reservoir solution includes a first electrolyte and the second reservoir solution includes a second electrolyte.

16. The method of embodiment 10, wherein the microfluidic device further includes a first electrode arranged in the first reservoir and a second electrode arranged in the second reservoir.

17. The method of embodiment 16, wherein method includes anionic analytes migrating from the first reservoir to the second reservoir, and: the first electrode is a cathodic electrode; the first reservoir solution is a cathodic reservoir solution; the first reservoir is a cathodic reservoir; the second electrode is an anodic electrode; the second reservoir solution is an anodic reservoir solution; and the second reservoir is an anodic reservoir.

18. The method of embodiment 16, wherein method includes cationic analytes migrating from the first reservoir to the second reservoir, and: the first electrode is an anodic electrode; the first reservoir solution is an anodic reservoir solution; the first reservoir is an anodic reservoir; the second electrode is a cathodic electrode; the second reservoir solution is a cathodic reservoir solution; and the second reservoir is a cathodic reservoir.

19. The method of embodiment 10, wherein the mixed analytes include biomolecules.

20. The method of embodiment 10, wherein the mixed analyte sample includes at least one of nucleic acids, carbohydrates, peptides, or proteins.

21. The method of embodiment 10, further including solidifying the gel solution.

22. The method of embodiment 10, wherein the gel is a sieving gel for resolving analytes in the sample.

23. The method of embodiment 10, wherein the gel is thermally responsive.

24. The method of embodiment 10, wherein the gel includes one or more of: agarose, polyacrylamide, a polymer sieving matrix, a capillary gel electrophoresis matrix, or a thermal gel polymer.

25. The method of embodiment 24, wherein the thermal gel polymer includes one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

26. The method of embodiment 10, further involving including buffer in the gel solution and/or the mixed analyte sample.

27. The method of embodiment 10, further including detecting separation of analytes of the mixed analyte sample in the channel.

28. The method of any one of embodiments 10-27, wherein reference particles are embedded into at least a portion of the microfluidic device adjacent to the channel.

29. The method of any one of embodiments 10-28, including indirectly detecting a wavefront of analyte migrating within the gel, which migrating results at least in part from analyte interaction with an electric field, by detecting movement of the tracking particles in response to collisions of analyte molecules in the wavefront with the tracking particles and/or deformations of the gel.

30. The method of embodiment 29, wherein the migrating results at least in part from analyte interaction with an applied electric current and/or an applied electric voltage.

31. A microfluidic device, including: a channel, configured to accommodate a mixed analyte sample mixed with a gel solution, the channel having a first end and a second end; embedded within at least a portion of a wall of the channel, reference particles; a first reservoir coupled to the first end of the channel, the first reservoir being configured to accommodate a first reservoir solution; a second reservoir coupled to the second of the channel, the second reservoir being configured to accommodate a second reservoir solution; a first electrode arranged in the first reservoir; and a second electrode arranged in the second reservoir; wherein the first electrode and the second electrode are configured to apply an electric field across the microfluidic device.

32. The device of embodiment 31, wherein device is configured for anionic analytes to migrate from the first reservoir to the second reservoir, and: the first electrode is a cathodic electrode; the first reservoir solution is a cathodic reservoir solution; the first reservoir is a cathodic reservoir; the second electrode is an anodic electrode; the second reservoir solution is an anodic reservoir solution; and the second reservoir is an anodic reservoir.

33. The device of embodiment 31, wherein the device is configured for cationic analytes to migrate from the first reservoir to the second reservoir, and: the first electrode is an anodic electrode; the first reservoir solution is an anodic reservoir solution; the first reservoir is an anodic reservoir; the second electrode is a cathodic electrode; the second reservoir solution is a cathodic reservoir solution; and the second reservoir is a cathodic reservoir.

34. The device of embodiment 31, wherein the sample includes at least one of nucleic acids, carbohydrates, peptides, or proteins.

35. The device of embodiment 31, wherein the gel is a sieving gel for resolving the sample.

36. The device of embodiment 31, wherein the gel is thermally responsive.

37. The device of any one of embodiments 31-36, wherein the reference particles are configured as device movement/drift control reference markers.

38. A method of assaying analyte migration in a liquid or gel contained in a device, the method including: contacting the migrating analyte with a plurality of tracking particles of average diameter less than 10 microns, the particles being incapable of interacting with the analyte by binding, adsorption, or chemical reaction; observing motion of some or all of the tracking particles by at least one optical, fluorescence, or other electromagnetic measurement technique; and using differing motion of the tracking particles to infer presence and/or concentration of the migrating analyte.

39. The method of embodiment 38, wherein the observing includes one or more of observing fluorescence, fluorescence lifetime, resonance energy transfer, phosphorescence, reflection, polarization, scattering, absorbance, chemiluminescence, or magnetic properties of some or all of the tracking particles.

40. The method of embodiment 39, further including detection of electromagnetic emission at more than one wavelength.

41. The method of embodiment 38, further including particle tracking, single-particle tracking or tethered-particle motion tracking.

42. The method of embodiment 38, in which the analyte migrating in the gel includes electrophoretic, dielectrophoretic, isotachophoretic, or sedimentation motion.

43. The method of embodiment 42, in which the analyte migrating in the gel includes electrophoresis.

44. The method of embodiment 38, wherein the gel is contained at least in part within a solidified gel.

45. The method of embodiment 44, wherein the solidified gel is a sieving gel for resolving migrating analytes.

46. The method of embodiment 44, wherein the solidified gel is thermally responsive.

47. The method of embodiment 44, wherein the solidified gel includes one or more of: agarose, polyacrylamide, a polymer sieving matrix, a capillary gel electrophoresis matrix, or a thermal gel polymer.

48. The method of embodiment 47, wherein the thermal gel polymer includes one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

49. The method of embodiment 38, further including observing the tracking particles by camera, digital camera, PMT, scanner, microscope, telescope, detector array, time-gated, chopped, frequency-modulated, wavelength-filtered, polarization-sensitive, Raman, Surface-enhanced Raman, high numerical aperture, color-sensitive, lifetime, FRET, FRAP, intensified, phosphorescence, resistivity, ellipsometer, or high-density CCD observation, in flow, on a surface, or in suspension.

50. The method of embodiment 38, wherein the tracking particles include nanoparticles, microparticles, or quantum dots.

51. The method of embodiment 38, wherein the tracking particles include one or more of a polymer, a metal (such as gold or silver), silica or glass, a magnetic material, or a lipid.

52. The method of embodiment 51, wherein the tracking particles include a polymer including a dextran, a polystyrene, an acrylate, a poly(ethylene glycol), a peptide, or a nucleic acid.

53. The method of embodiment 38, wherein a temperature of one or more elements of a system including the device is controlled.

54. The method of embodiment 38, further including assaying more than one migrating analyte, which migrating analytes are separated by the migration, and the method includes using differing motion of the tracking particles to infer presence and/or concentration of the more than one migrating analyte.

55. The method of any one of embodiments 38-54, further including carrying out the analyte migrating assay in a device in including reference particles substantially equivalent in size and detection capability to the tracking particles with which the analyte(s) is contacted, wherein the method further includes controlling for device movement when calculating the differing motion of the tracking particles by detecting movement of one or more reference particles.

56. An improved method of thermal gel electrophoresis (TGE), the improvement including: employing particle motion tracking (PMT) to detect and/or quantify label-free analyte(s) being subject to the TGE.

57. A label-free analyte separation strategy based on gel electrophoresis, substantially as disclosed herein.

58. A method for separating and detecting and optionally quantifying two or more biomolecule species in a mixed sample, the method including thermal gel electrophoresis and particle motion tracking substantially as described herein.

59. A method of improving analyte detection in a gel electrophoresis device, including embedding or otherwise attaching reference particles in/on the gel electrophoresis device.

60. The method of embodiment 59, wherein the embedded/attached reference particles are used to correct for device drift and/or to improve signal quality of movement of tracking particles used in the method.

### (XIV) Examples

Example 1: Exploiting collision-influenced motion within a high-viscosity thermal gel

Although Brownian motion generally describes particles in a liquid, this example described a system that harnesses such motion of particles while entrapped in a high-viscosity thermal gel.

Studies were conducted where fluorescent beads (200 nm) were cast into thermal gel along with a model analyte, fluorescein (500 nM), and analyzed by thermal gel electrophoresis (TGE). A control analysis was first conducted to measure the direct fluorescence of fluorescein to determine its migration time (FIG. 15, DF). This analysis also confirmed that beads did not migrate in the electric field because they were trapped in the solidified thermal gel.

A second run was then conducted that tracked motion of beads embedded in the gel at the detection point (FIG. 15, PM). A change in motion was observed as analytes passed, including a peak with a similar migration time as the fluorescence control run. Interestingly, other peaks also appeared in the analysis that were not present in the control run.

These data demonstrate that motion of beads detected species regardless of whether they were fluorescent. The other peaks were attributed to electrolyte bands that migrated during TGE.

Example 2: Integrating Particle Motion Tracking into Thermal Gel Electrophoresis for Label-Free Sugar Sensing

Bioanalytical sensors are adept at quantifying target analytes from complex sample matrices with high sensitivity, but their multiplexing capacity is limited. Conversely, analytical separations afford great multiplexing capacity but typically require analyte labeling to increase sensitivity.

This example describes development of a separations-based sensor to sensitively quantify unlabeled biomolecules (exemplified with polysaccharides) using insoluble particle motion tracking within a microfluidic electrophoresis platform. Carboxymethyl dextran (20 kDa) was spiked into Pluronic thermal gel along with fluorescent nanoparticles (200 nm diameter) and loaded into single-channel microfluidic devices. Upon voltage application, the soluble sugar enriched into a concentrated band that induced motion of the insoluble particles (a.k.a., bead displacement) as the concentrated band passed. Bead displacement was tracked over time to produce electropherograms, where peak areas (the area under the charted curve at a peak) were directly proportional to analyte concentrations.

Key studies herein established ranges of acceptable operating conditions (e.g., gel concentration, temperature) to characterize how the temperature-dependent rigidity of thermal gel influenced the analysis. Data processing strategies were evaluated to identify conditions (e.g., exposure intervals, particle averaging, motion directionality) to maximize sensitivity. The quantitative response of the method was evaluated over a broad concentration range (0.5-5000 nM) where detection limits were found to be 520 μM for the 20 kDa sugar, providing a 10⁶-fold superior mass LOD than a gold standard UV-Vis absorbance method. Studies into the detection mechanism found that sensitivity was dependent on the molecular weight of the sugar (more generally, the analyte being studied), as larger sugars produced greater responses. Collectively, these studies establish exemplary best practices for integrating particle sensing into thermal gel separations for label-free polysaccharide quantitation.

At least some of the material described in this Example, and more generally in this patent document, may have been disclosed in Cornejo & Linz (ACS Sens. 2025 Jan. 24; 10 (1): 204-212. doi: 10.1021/acssensors.4c02042. Epub 2025 Jan. 3).

Background & Context: The recent emergence of particle motion-based sensors has led to improved characterizations of biological samples. For example, nanoparticle tracking analysis (NTA) utilizes particle motion to determine the size distributions of bioparticles. In such methods, Brownian motion of individual particles is monitored to determine particle diffusion coefficients, which are then converted into particle sizes. ¹ NTA has been used to characterize extracellular vesicles, viruses, drug delivery nanoparticles, and protein aggregates. ²⁻⁴ Biosensing by particle motion (BPM) extends particle motion-based sensing to the analysis of biomolecules. Particles functionalized with biological recognition elements (e.g., antibodies, oligonucleotides) are first tethered to a surface. A biological sample is then introduced, causing target biomolecules to selectively bind to the particles, consequently altering their Brownian motion. ⁵ By tracking the displacement of individual particles over time, analyte binding events can be monitored to quantify analytes with pM-mM limits of detection (LODs) and to study dissociation kinetics of binding interactions. ⁶⁻⁸ The abilities of NTA and BPM sensors to characterize biological nanoparticles and biomolecules with high sensitivity highlight the utility of particle motion detection. However, further advancements are still needed to increase multiplexing capacity of the analysis, expand the scope of biomolecules that can be measured, and eliminate requirements for external reagents (e.g., stains, antibodies) for true label-free analysis.

Separations techniques are an alternative for characterizing biological samples. Techniques such as capillary electrophoresis and liquid chromatography can quantify diverse biomolecules with great multiplexing capacity but consume relatively large volumes of reagents and have limited sensitivity compared to sensors. To overcome these issues, separations-based sensors have been developed that couple microchip electrophoresis to an electrochemical sensor. ^{9, 10} This dual approach enables analytes in complex mixtures to first be separated before arriving at a sensor to provide unbiased quantitation of each species. A limitation, however, is that analytes must be inherently electroactive to achieve label-free detection. This requirement precludes detection of most biomolecules. Consequently, analyte capture or derivatization steps must be incorporated into most analytical workflows to render analytes either electroactive or fluorescent. ^{11, 12} This sample preparation requirement increases complexity of the method as well as its time and cost.

Additionally, not all biomolecules are amenable to labeling. Sugars, in particular, pose a significant challenge because only reducing sugars can undergo derivatization. ¹³ Even with those, labeling procedures for reducing sugars require high heat, caustic solvents, and lengthy preparation times along with subsequent purification and lyophilization steps. ¹⁴⁻¹⁶ These time- and cost-intensive steps hinder analyses of sugar-based biologically and pharmaceutically significant biomolecules (e.g., heparin). There is a critical need for a label-free sugar analysis platform that obviates elaborate sample preparation steps and expedites analyses.

Integrating particle motion sensing with microfluidic electrophoresis has the potential to create a separations-based sensor for the analysis of sugars (and other biomolecules). Such an approach would provide both the high multiplexing capacity of electrophoresis and the sensitive label-free detection of particle tracking. However, combining these techniques poses significant technical challenges. Particles must be included at the detection point within the separation space to track passing analytes. These particles must be free to move in response to migrating analytes but restrained so they do not migrate themselves in the applied electric field. These constraints preclude tethering particles at the detection point because they would stretch along the electric field and not respond to migrating analytes. An alternative approach is needed to confine particles in place during a separation.

Thermal gel electrophoresis (TGE) is well suited to incorporate detection particles into its microfluidic separations platform. TGE employs thermal gels (that is, water-soluble polymers that possess the characteristic of undergoing a change in viscosity in response to changing temperature; Pluronic thermal gel is an example) as the media for electrokinetic analyses. Thermal gels are composed of block copolymer micelles that can reversibly assemble into a crystalline lattice. ^{17, 18} Micelle packing density depends on the temperature and ionic strength of the surrounding medium, which can be controlled to dynamically adjust gel viscosity. ¹⁹ A key feature of previous TGE reports is an inline analyte preconcentration and separation. ²⁰⁻²² Biomolecules are cast in thermal gel and loaded throughout single-channel microfluidic devices. Analytes enrich together and then separate apart upon voltage application, without requiring a sample injection or user intervention. This simple technique streamlines analyses and has been used for high-sensitivity measurements of proteins and miRNAs. ²⁰⁻²² TGE is also effective at analyzing larger particles. A report demonstrated that cells in thermal gel can be enriched, washed, and subsequently collected by regulating the temperature in microchannels to control gel viscosity. ²³ Higher temperatures pack thermal gel micelles more tightly to non-covalently confine cells in the solidified gel lattice. Concurrently, small molecule cell-staining dyes pass through the solid gel to be washed away. Cells can then be released by reducing temperature to disperse thermal gel micelles and re-impart mobility to the cells. Given the unique ability of thermal gels to separate soluble molecules and also reversibly confine insoluble cells, thermal gels are an excellent material to incorporate particle motion tracking (PMT) detection into an electrophoresis platform for the analysis of biomolecules.

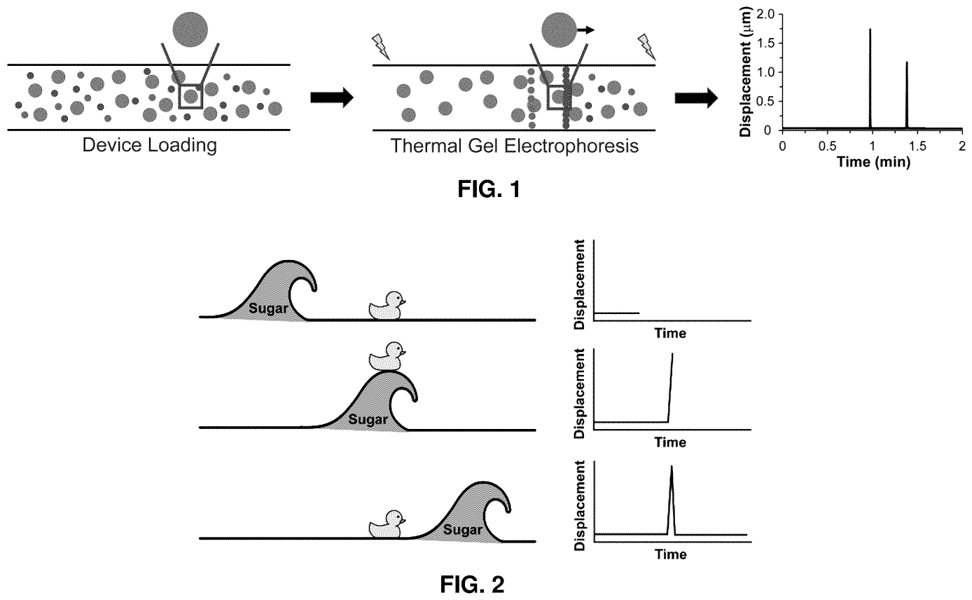

This report describes the development of a separations-based sensor that integrates PMT into TGE for the label-free quantitation of polysaccharides. Tracking beads and sugars were spiked into liquid-phase thermal gel that was then loaded throughout a single-channel microfluidic device (FIG. 1). Thermal gel was solidified to immobilize the particles in a lattice of packed gel micelles within the channel. Voltage was applied to induce electrokinetic enrichment and separation of the sugars. As the bands of sugar collided with the beads, their baseline motion was altered to provide a quantitative response (FIGS. 1, 2). The studies herein demonstrate feasibility for integrating PMT with TGE and establish best practices for quantitative data processing. This approach obviated the need to fluorescently label sugars and perform subsequent washing and purification steps. This label-free analysis has the potential to provide universal detection of diverse biomolecules in complex biomedical samples in future applications.

### Experimental

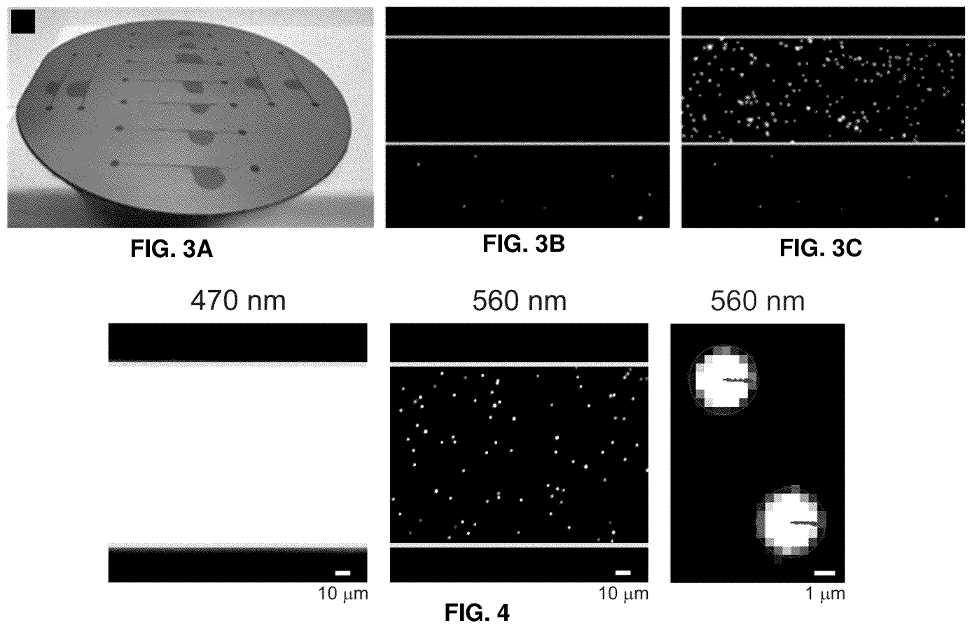

Device Fabrication: Photolithography was conducted using SU-8 (Kayaku Advanced Materials, Westborough, MA) to fabricate a master wafer containing 11 channel features on a 4-inch silicon wafer (University Wafer, South Boston, MA). The final feature dimensions measured 120 μm wide, 20 μm tall, and 30 mm long. Devices were prepared by casting a 7:1 mixture of degassed PDMS elastomer base: curing agent onto the master wafer and baking for 2 h at 70° C. In devices that employed reference beads, FluoSpheres™ were first mixed into a separate aliquot of PDMS (1×10⁵ beads/μL). A thin layer (≤1 μL) of this bead-containing PDMS was then spotted onto the master wafer near the detection point (20 mm) of each feature (FIGS. 3A-3C). The spotted bead-PDMS was semi-cured onto the master wafer for 20 min at 70° C. Blank PDMS was then poured onto the wafer as usual and placed in the oven for 2 h at 70° C. This approach cured both PDMS layers together while preserving reference beads at the detection point and preventing them from becoming embedded over the channel. Devices were then diced from the cured PDMS and reservoirs created using a 3 mm biopsy punch (Ted Pella, Redding, CA). Each device was reversibly sealed onto a glass microscope slide (AmScope, Irvine, CA) to form enclosed channels.

Data Processing Considerations: Bead tracking images were processed in FIJI using TrackMate 7 (Ershov et al., Nat Methods 19 (7): 829-832, 2022). The Laplacian of Gaussian filter was applied in the software to better identify the center of beads with sub-pixel localization and prevent distortion during tracking. To ensure the software precisely tracked individual particles, a low density of tracking beads was selected (1×10⁵ beads/μL) that limited overlap (FIG. 4). Beads selected for processing were chosen manually to avoid any overlapping particles and with preference given to those in a vertical line (i.e. similar x-axis coordinates). Beads near the channel walls were excluded to eliminate the potential of being inadvertently identified as reference beads. Additionally, low-intensity beads were omitted from processing to prevent tracking difficulties, and high-intensity beads were omitted to ensure bead aggregates were not included to avoid potential measurement bias. Selected tracking beads were then vetted to confirm their viability before processing fully. Each device was subject to a post-run analysis in liquid-phase thermal gel to elicit bead migration. Tracking beads that migrated freely were confirmed to be viable. Beads that did not migrate were excluded from the data set because their adhesion onto the glass biased measurements. This issue was not prevalent, as <5% of beads required exclusion from data sets. Optimal signal-to-noise (S/N) in PMT electropherograms was attained by measuring the positions of six tracking beads within the channel and six reference beads embedded in the PDMS device. S/N was determined by ratioing the peak height in the position data to the average baseline height in the first 30 s of the analysis.

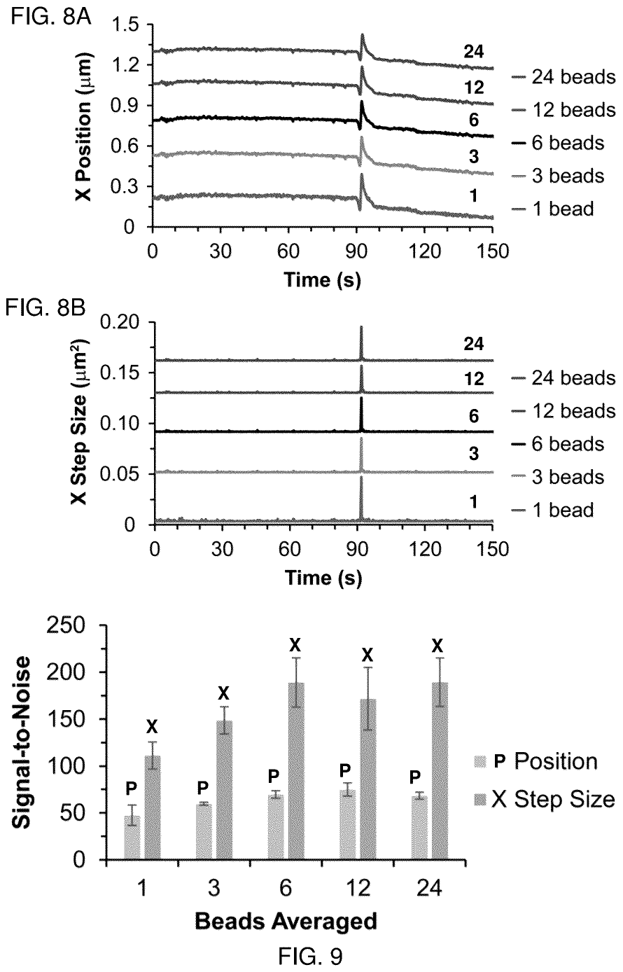

Absorbance Measurements: UV-Vis absorbance measurements were used to characterize the unlabeled sugar and compare against data collected with TGE-PMT. CM-dextran was diluted in ultrapure water to prepare calibration standards 1-500 μM. Each standard was analyzed in triplicate using a Shimadzu UV-1800 Spectrophotometer (West Chicago, IL). The response from 200-210 nm was averaged to mitigate variability between replicate scans and obtain the final absorbance value. The calibration curve across this concentration range is shown in FIGS. 8A-8B. The trendline equation is in Table 2. The detection limits with absorbance were found to be 150 nM.

Microfluidic Device Operation: Single-channel microfluidic devices were used for the studies herein to maximize simplicity and minimize cost. A master mold was fabricated from SU-8 using standard photolithography and polydimethylsiloxane (PDMS) devices replicated from it, as described in previous reports. ^{20, 24} Details on device fabrication (FIGS. 3A-3C) and sources of reagents are provided. Thermal gel stock was prepared by dissolving 33.3% (w/v) Pluronic F127 in 30 mM tris-HCl (pH 8.0) and placing in a refrigerator (4° C.) for 1-2 hrs. Samples were prepared by combining FluoSpheres™ with carboxymethyl dextran (CM-dextran) and casting the solution into thermal gel at a 1:9 ratio. Final samples contained 30% (w/v) F127, 1×10⁵ beads/μL FluoSpheres™, and variable concentrations of CM-dextran. Microfluidic devices were filled on ice with sample-containing thermal gel. This low temperature dispersed the thermal gel micelles to maintain the gel in its liquid state, which enabled CM-dextran and beads to be stochastically loaded throughout the channel (FIG. 4). Warming the device to room temperature packed thermal gel micelles into a solid lattice to immobilize beads for analysis. Excess gel was then vacuumed from the reservoirs. For PMT optimization experiments, the cathode reservoir was filled with 400 mM glycine and 5 mM tris-HCl (pH 7.5) and the anode reservoir filled with 30 mM tris-HCl (pH 8.0). The same electrolytes were employed for separation experiments, but 2 mM tricine was also added to the cathode reservoir and 200 mM ammonium acetate to the anode reservoir.

Loaded devices were placed on the temperature-controlled stage of an AZ100 epifluorescent microscope (Nikon Instruments Inc., Melville, NY). Images were acquired during analyses with an ORCA-Fusion sCMOS camera (Hamamatsu Corp., Bridgewater, NJ) using 150 ms exposures. Excitation light was produced by a SOLA Light Engine (Lumencor, Beaverton, OR) using a Texas Red filter cube (560/630 nm) for particle tracking analyses or a FITC filter cube (470/525 nm) for direct fluorescence analyses of FITC-CM-dextran. The microscope was focused at a detection point 20 mm from the cathode reservoir using 32× magnification. Voltage (1.5 kV) was applied across the microchannel with a high-voltage power supply (UltraVolt Inc., Ronkonkoma, NY) controlled by a custom LabVIEW program (National Instruments, Austin, TX). After TGE-PMT analyses finished, a secondary analysis of each device was performed to vet the tracking beads. The stage temperature was reduced to 15° C. to liquify the gel and the voltage increased to 2 kV. This induced bead migration except in beads adhered onto the device surface or reference beads embedded in the PDMS. These stuck beads were excluded from subsequent data processing.

Data Processing: Series of fluorescence images were processed using FIJI. ²⁵ Direct fluorescence measurements integrated the intensity versus time to create electropherograms. Particle motion measurements used the TrackMate 7 program within FIJI to track bead positions versus time. ²⁶ Specific criteria for particle selection are discussed in “Data Processing Considerations” in the SI. Multiple data processing approaches were evaluated to determine best practice for TGE-PMT. Consistent between all methods, TrackMate software was used to measure the x and y coordinates for each selected bead in every frame of the image series. Initial studies measured the mean squared displacement (MSD) of each bead in each frame relative their initial positions using the expression [(xₙ−x₀)²+(yₙ−y₀)²]. ²⁷ Subsequent data processing considered the squared displacement along the x and y axes independently using the expressions (xₙ−x₀)² or (yₙ−y₀)². Frame-to-frame displacement was also evaluated by measuring the squared step sizes using (xₙ−xₙ₋₁)². However, referencing to the origin distorted the electropherograms and squaring the data obscured the direction of particle motion. Ultimately, optimal data processing was found to consider either x-axis position (xₙ) or x-axis step size (xₙ−xₙ₋₁). Both data processing interpretations reduced noise and maximized sensitivity of the analysis. Once TGE-PMT electropherograms were generated, peak areas were calculated from the traces using Chromophoreasy. ²⁸ Error bars in all figures depict ±1 standard deviation from n=3 replicates. Note that in most figures, error bars are smaller than the data point markers.

Reagents: Pluronic F127 (aka Poloxamer 407) and ammonium acetate were obtained from Millipore Sigma (Burlington, MA). FluoSpheres™ carboxylate-modified microspheres (0.2 μm diameter), glycine, tricine, and 1 M tris-HCl were purchased from ThermoFisher (Waltham, MA). Three fluorescein isothiocyanate-carboxymethyl dextrans (FITC-CM-dextran) were obtained from TdB Labs (Uppsala, Sweden) with molecular weights of 4 kDa (product sheet specifies a range from 4-6.5 kDa), 20 kDa (ranging from 20-30 kDa), and 40 kDa (ranging from 40-60 kDa) along with unlabeled 20 kDa CM-dextran (ranging from 20-30 kDa). All solutions were prepared using 18.2 MΩ·cm ultrapure water from an ELGA LabWater Purelab Classic (High Wycombe, UK). Polydimethylsiloxane (PDMS) was purchased from Ellsworth Adhesives (Germantown, WI).

### Results and Discussion

### Characterizing Bead Motion in Thermal Gels:

Particle motion sensing presents an interesting approach for label-free detection in electrokinetic separations. However, feasibility of this strategy has never been demonstrated. In the foundational studies herein, CM-dextran (20 kDa) was selected as the model analyte because of its similar anionic charge and size as polysaccharide pharmaceuticals (e.g., heparin) and its use as a drug delivery agent. ²⁹⁻³¹ A FITC conjugate of the sugar was used during method development to enable comparisons between PMT and direct fluorescence detection to validate the described sensing scheme. Fluorescent carboxylated polystyrene beads (200 nm diameter) were used for particle tracking. Their anionic charge was expected to be repelled by the anionic sugar as it migrated through the channel to provide good response in PMT. Beads were loaded stochastically throughout single-channel microfluidic devices at a density that maximized the number of beads present while minimizing bead overlap. This approach enabled the responses of multiple beads to be averaged while also minimizing errors in particle tracking due to overlapped beads. Beads were not expected to interfere with the separation, as they only occupied 0.0001% of the channel volume at the selected bead density.

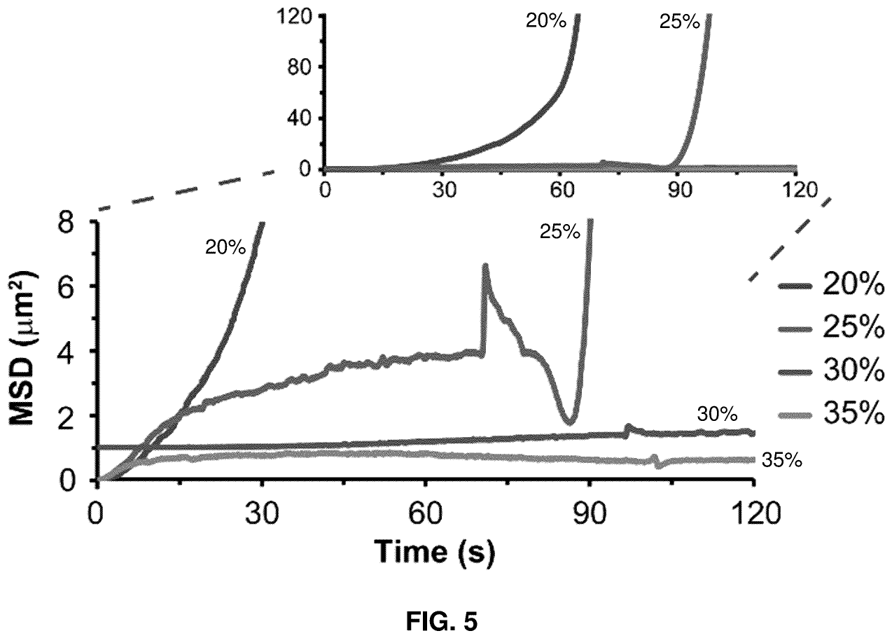

Initial studies were conducted to identify conditions amenable to PMT detection. Thermal gels composed of 20-35% (w/v) Pluronic F127 were evaluated to determine the range of polymer concentration where beads remained embedded in the gel but still responded to the migrating sugar band. The supporting electrolytes for TGE (see Methods) were selected to enrich the FITC-CM-dextran into a low-volume, high-concentration band. The positions of beads were measured during the analysis to track their MSD, which reflects the instantaneous position of each bead relative to its starting position. ²⁷ Results from the study found that gels containing 30% and 35% F127 exhibited similar responses (FIG. 5). A flat baseline was observed until a small peak appeared at a similar migration time as control analyses that directly measured the FITC-sugar. In 25% F127 thermal gel, the bead MSD peak was significantly larger although the baseline was less stable. The increased response was attributed to the lower polymer concentration producing fewer thermal gel micelles to hold the beads in place. Thus, beads were able to move to a greater degree in this less confining gel. Although sensitivity to the sugar was high in this 25% thermal gel, beads no longer remained stationary after the sugar peak passed and instead began to migrate through the channel (FIG. 5, inset). This suggests that the analyte irreversibly disrupted thermal gel micelle packing such that the gel lost rigidity and could no longer confine the beads. Although the sugar could be measured, this instability would preclude detection of other peaks in a separation. In the 20% F127 gel, beads began to migrate immediately following voltage application. Beads in this 20% gel did not remain in the field of view for the duration of the analysis because thermal gel micelles were not packed densely enough to prevent the beads from migrating in the electric field. However, the overall results from this study found that PMT is viable in thermal gels containing 25-35% F127, which aligns with gel concentrations used in previous TGE reports. ^{17, 20, 22} Beads remained embedded in the gel at those concentrations while responding to the analyte band. 30% F127 gel was used in subsequent studies despite its lower signal than the 25% gel, because of its more stable baseline and ability to prevent bead migration.

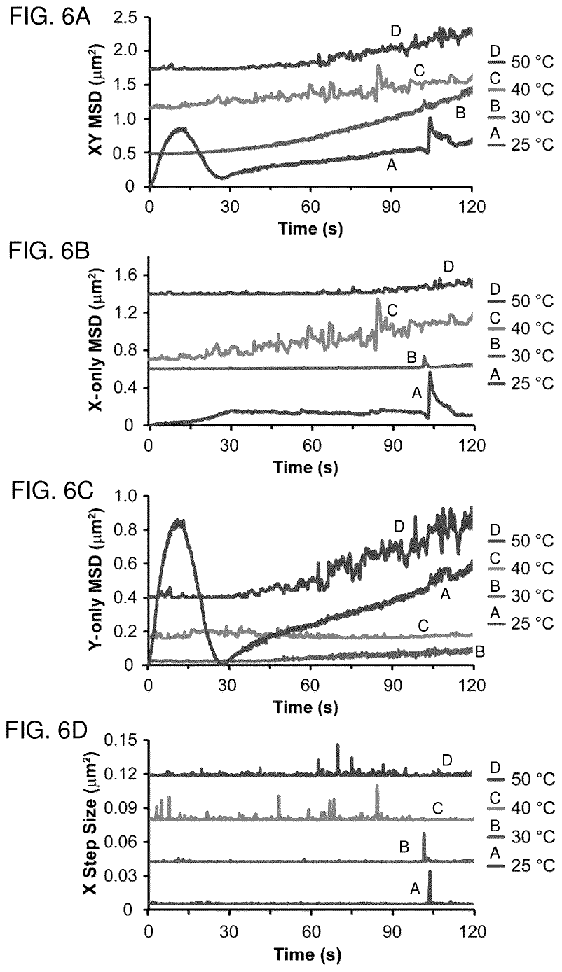

In addition to polymer concentration, the viscosity of thermal gels is also significantly impacted by temperature. Thus, a temperature study was conducted to evaluate the viability of PMT from 20-50° C. in 30% F127 to determine the accessible operating range. Results from this study found that beads migrated freely at 20° C. and rapidly exited the field of view. Runs at 25° C. exhibited variable baseline bead motion but responded intensely to the passing sugar (FIG. 6A). A similar trend was observed at 30° C., but the peak was smaller than at 25° C. because the more densely packed thermal gel micelles inhibited particle motion. ¹⁷ Interestingly, peaks in these runs returned to baseline after the analyte band passed. This finding indicates that beads did not remain in the new position or continue to drift down the channel. Instead, they initially moved downstream in response to the passing analyte but then rebounded back to the original position once the analyte band migrated away. This provides insight into the mechanism of bead movement in thermal gels. Similar responses were not observed at 40 and 50° C. because excessive noise obscured the analyte. This behavior was attributed to higher thermal energy increasing Brownian motion of the beads. These results suggest that increases in random motion at elevated temperatures are more dominant than the higher thermal gel viscosity at these temperatures, ³² resulting in a noisier separation.

Both the gel concentration and temperature characterization studies demonstrate feasibility for incorporating bead tracking into microfluidic gel separations. However, the data quality was low due to small analyte peaks and noisy baselines. This led us to question if MSD was the appropriate parameter to assess bead motion. MSD considers both x-axis and γ-axis motion of particles to monitor their random Brownian motion. ²⁷ However, bead motion in TGE is not random. Beads move in response to passing analyte bands, which travel along the x-axis. ³³ Therefore, bead displacement along the x-axis is expected to correlate directly to the analyte whereas motion in the y-axis may only contribute to noise in the analysis. To evaluate this supposition and determine best practice for data processing in TGE-PMT, the data sets from the temperature study were reprocessed. The same images were processed to consider x-axis and y-axis MSD (FIG. 6A), x-axis-only squared displacement (FIG. 6B), and y-axis-only squared displacement (FIG. 6C). Using the same data to assess processing practices controlled for inter-analysis variability between replicate devices.

Data sets using x-y, x-only, and y-only positions all responded to the passing analyte band but to significantly different degrees. Traditional x-y MSD exhibited a response to the band at 25 and 30° C. but also had random peaks and baseline drift (FIG. 6A). Considering motion only in the x-axis—along which the analyte band travels—produced only a single peak in response to the band at 25 and 30° C. (FIG. 6B). The x-only signal was more intense than the MSD signal, indicating greater sensitivity. Additionally, the random peaks and baseline instability in the MSD data were no longer present when exclusively considering x-axis motion. These results reveal that x-axis squared displacement provides a less noisy, more selective response to the analyte band. This interpretation is validated when considering the response in the y-axis (FIG. 6C). Although some response from the sugar may be present in the data at 25° C., the random peaks and baseline deviations primarily arose from the y-direction. Given that MSD utilizes both x-axis and y-axis bead positions, the noise in the MSD primarily originates from random motion in the y-axis.

Although x-axis squared displacement provided more meaningful data than x-y MSD, further improvement clarity in the signal was sought. Conventional squared displacement values consider all positions relative to the origin. However, to maximize sensitivity in a separation, the step size between successive images may be more impactful to only consider the instantaneous response as analyte passes. ³⁴ To evaluate this, the same data sets from the temperature study were processed to measure the instantaneous squared step size in the x-axis. The resulting peaks were significantly sharper and more intense (FIG. 6D) than when scaling to the origin (FIG. 6B). Additionally, noise in the baseline was removed when considering only instantaneous bead motion. This higher S/N is key to improving detection limits in analytical separations. This benefit is exemplified in the analyses at 40 and 50° C. While no obvious peaks could be identified using the x-only squared displacement relative to the origin (FIG. 6B), sharp peaks were observed when considering instantaneous x-axis squared step size (FIG. 6D). Multiple other peaks also appeared in these high-temperature analyses, so the data remained noisier than at lower temperatures, but clear improvements were gained. This higher quality data was able to reveal that temperature affected the migration time of the band, as higher temperatures promoted faster migration due to the increased thermal energy in the system, which is consistent with previous literature. ^{21, 35, 36}

Overall, these studies determined that data quality is improved by measuring bead positions exclusively in the direction of migration along the x-axis. While x-only squared displacement was promising, further evaluations revealed that flatter baselines and more consistent results between replicates were obtained without subtracting the origin coordinates and not squaring the values. This simplified data processing also now enabled assessment of bead directionality, which provides additional information (discussed in the next section). Thus, subsequent studies only considered the unaltered x-axis position. Furthermore, squared step size was found to be a viable processing method that provided high sensitivity. Because of their good performances, both position and step size data processing methods were translated to subsequent studies.

### Establishing Best Practices in Image Acquisition and Data Processing

Having established feasibility of particle tracking detection in gel separations, optimal conditions for imaging and best practice for data processing were next determined. Success of PMT hinges on capturing bead motion at an appropriate interval to effectively measure the response of beads to passing analyte bands. A careful balance must be struck when selecting an interval. Longer exposures over-average signal from beads and mask motion, which reduces sensitivity. Conversely, shorter exposures capture less signal from beads, which may hinder the particle tracking software from reliably monitoring bead positions. Additionally, short exposures also necessitate that beads be tracked through more frames, which increases computational costs and data processing times.

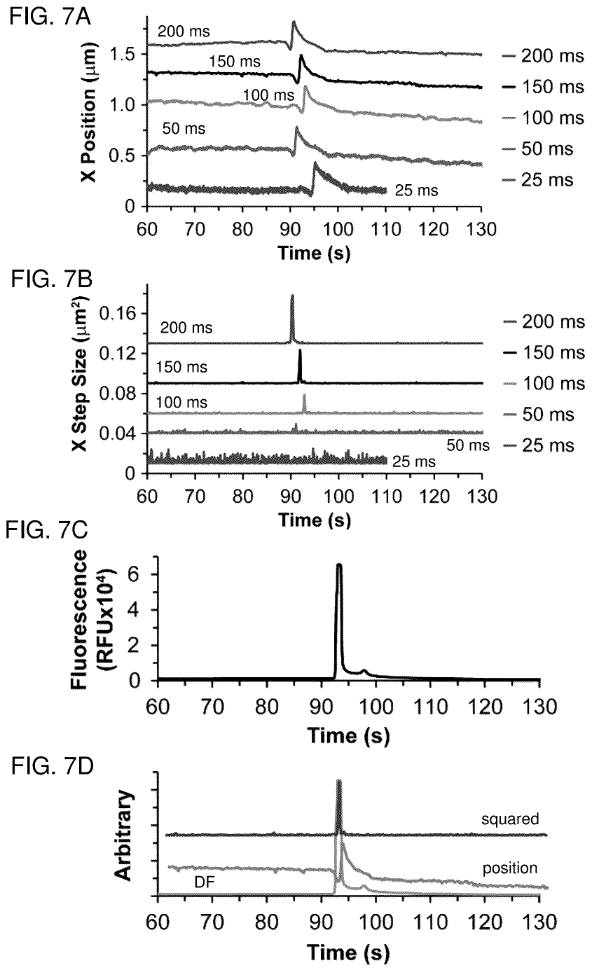

A study was conducted to determine the optimal image exposure times for TGE-PMT. FITC-CM-dextran and tracking beads were cast into 30% F127 thermal gel and analyzed at 30° C. Images were acquired with 10-200 ms exposures (FIGS. 7A-7D). Shorter exposures are typically preferred in BPM because they better capture subtle bead motions. Thus, initial TGE-PMT analyses used 10 ms exposures, but particles were not reliably tracked throughout the runs due to their low intensities. Beads were missed between frames, which led to inconsistent data sets. Analyses using 25 ms exposures were tracked without issue. However, x-axis position data (FIG. 7A) at this short exposure time had relatively large noise while x-axis squared step size (FIG. 7B) had excessive noise that precluded detection of the analyte. As exposure times increased, S/N of the electropherograms increased as well. Beads imaged at 150 and 200 ms exposures produced the strongest signals for the analyte band and were easily tracked throughout the analysis. Although this is contrary to best practice in BPM, larger perturbations of beads between successive images from the migrating analyte produced larger PMT signal. Ultimately, 150 ms was determined to be optimal. Beads were readily identified by the particle tracker program while exhibiting high sensitivity to passing analyte bands and minimizing risks of over-averaging. Computational costs were also reduced at 150 ms than with shorter intervals, which is important for data management in subsequent applications. Note that in applications employing higher electric field strengths, faster acquisitions may be needed to ensure adequate sampling across the peaks.

Direct fluorescence analyses of FITC-CM-dextran were used to validate the response of PMT. Electropherograms were acquired at the excitation wavelength of the FITC label (FIG. 7C). This data showed an intense peak for the sugar at a similar migration time as that observed in PMT. This result confirms that the peak observed by the tracking beads arises from the migrating sugar. The peak width from the direct fluorescence analysis (FIG. 7C) was wider than with x-axis bead squared step size (FIG. 7B). However, the step size peak width correlated well with the direct fluorescence peak width at half height (FIG. 7D). This result indicates that bead step size is most sensitive to the middle-most concentrated-region of the migrating analyte band. Peak shapes were similar for both squared step size PMT and direct fluorescence. However, x-axis position PMT exhibited a unique profile. The bead position data shows that beads initially move upstream in the channel before being propelled downstream and ultimately returning to the original baseline position (FIG. 7A). This response is similar to that of ocean buoys moving in response to passing waves. Buoys follow circular trajectories as waves pass, where they are first drawn out to sea before being pushed towards shore and then returning to their original position. ³⁷ Although numerous differences exist between ocean waves and electrophoretic separations, this interesting observation suggests that beads embedded in thermal gel exhibit buoy-like responses to passing analyte “waves”. The rates of upstream motion—both before and after the passing analyte wave—are similar but are 6-fold slower than the downstream motion, which indicates that the flow is significantly more intense than the ebb. Returning to the x-position data, the peak width initially appears wider than that of the direct fluorescence analysis. But a closer look at the direct fluorescence electropherogram (FIG. 7C) shows that the fluorescence never quite returns to baseline before a small second peak appears, likely from a high-mass contaminant in the FITC-CM-dextran. The bead position data (FIG. 7A) exhibits this same intermediate region along with a peak shoulder that aligns well with the contaminant peak (FIG. 7D). Although determining the identity of the contaminant was beyond the scope of this PMT development study, detecting a trace-level contaminant is encouraging because it shows the high sensitivity of TGE-PMT. Resolution was lower than with direct fluorescence, but the ability to detect this species without the need for fluorescence labeling is an important advancement in analytical characterizations of sugar formulations.

Signal averaging is another important parameter to consider while establishing best practice for TGE-PMT data processing. Averaging the responses of more beads is expected to improve S/N of the analysis, but the optimal number must be determined. A key consideration for this analysis is how far apart the beads are spaced in the channel. Ideally, all beads would have identical x-axis positions—only spaced apart along the y-axis—so that the analyte band collides with all beads simultaneously to synchronize their responses. If beads dispersed along the x-axis are averaged together, upstream beads may shift in response to the passing analyte while downstream beads remain stationary. This over-averaging would dilute signal and reduce sensitivity.

A study was conducted to determine optimal signal averaging in an analysis of FITC-CM-dextran. Within a single device, numerous beads at the detection point were tracked, and the x-axis position (FIG. 8A) and instantaneous x-axis squared step size (FIG. 8B) were calculated. Results from tracking a single bead in the middle of the channel yielded a clear peak to the passing analyte band. Averaging values together from three beads—located along a vertical axis—reduced noise in the baseline and increased S/N of the analysis (FIG. 9). S/N further improved by averaging tracking data from six beads. Performance then plateaued, as no significant benefit was observed by interrogating 12 or 24 beads. The diminished returns when averaging more beads was attributed to bead dispersement. As more beads were included in the data set, it necessitated beads being selected that had more distance between them (Table 1). This x-axis scatter led to unsynchronized responses between upstream and downstream beads, which hindered the signal. Ultimately, it was determined that averaging six beads using either x-axis position or x-axis squared step size was optimal to maximize S/N while also minimizing computational processing costs.

TABLE 1
Maximum distance between beads within
an analysis (n = 3 devices).

| Number of Beads Averaged | Max Distance Between Beads (mm) |
|---|---|
| 3 | 13 ± 2 |
| 6 | 25 ± 5 |
| 12 | 60 ± 10 |
| 24 | 117 ± 8 |

### Quantitative Label-Free Analysis Using In-Gel Particle Tracking

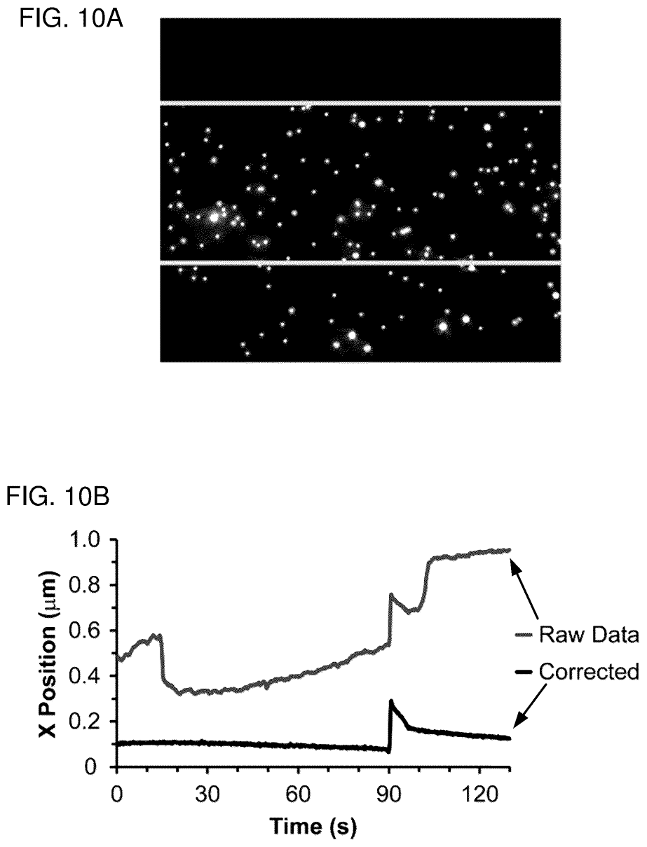

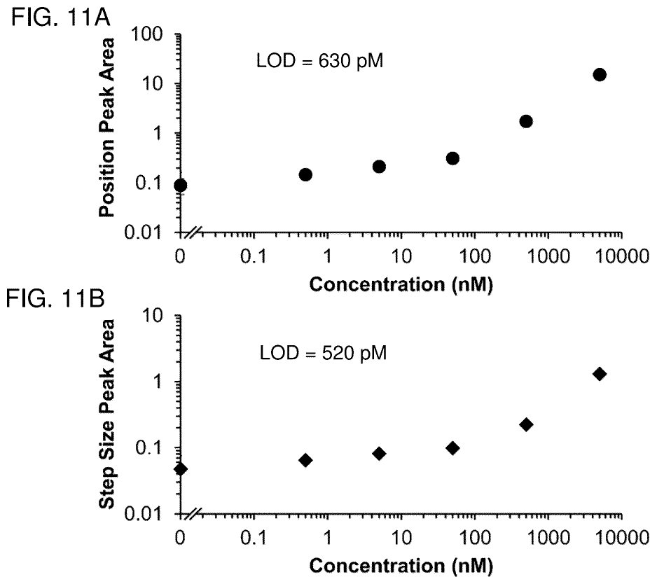

The work above established the integration of PMT detection into TGE analyses. However, during these optimization studies, random baseline drift was occasionally observed, which diminished reproducibility between replicate analyses. Although this noise did not interfere with preliminary demonstrations, efforts were made to mitigate it before pursuing quantitative studies. The source of the noise during analyses was ultimately determined to arise from small shifts of the microfluidic devices. During runs, the entire device would occasionally move a few microns. Although these small positional drifts are inconsequential when integrating signal across a 120-mm wide channel—as with standard fluorescence detection—a micron-long drift is larger than the 0.2 mm diameter of the beads being tracked. This led to baseline drift and random noise spikes during some analyses. It was therefore determined that reference beads (aka reference particles) should be incorporated into the analysis to correct for device drifting. This was achieved experimentally by adding beads into PDMS and spotting them near the 20-mm detection point during device fabrication (FIG. 10A). The same FluoSpheres™ used as tracking beads were also used as reference beads to enable both sets of particles to be tracked simultaneously under the same experimental conditions and image processing settings. Embedding beads in the device itself enabled device drift to be tracked independently from the separation occurring in the channel.

Analyses of 500 nM FITC-CM-dextran were conducted using devices prepared with reference beads. Image data sets were processed to measure the positions of tracking beads (inside the channel) and reference beads (outside the channel) throughout the run. The average position of the reference beads was then subtracted from the position of each tracking bead during data processing. Although many devices did not benefit from this additional processing, this approach corrected for device drift in problematic analyses, which flattened the baseline and removed random noise spikes (FIG. 10B). Reference beads were ultimately found to improve reproducibility between analyses and were thus incorporated into all subsequent studies.

Having established best practices for experimentally conducting TGE-PMT and data processing, the quantitative response of the integrated technique was next evaluated. The model analyte used in initial studies—FITC-CM-dextran—is fluorescently labeled and can be directly measured with fluorescence. This provided helpful comparisons while developing the technique. However, the true power of TGE-PMT is the ability to analyze unlabeled analytes. To demonstrate viability for label-free sensing, quantitative studies were performed using unlabeled CM-dextran. Pilot quantitation studies demonstrated good performance for position peak areas, but squared step size peak areas were more variable. It was determined that simply considering step size—without squaring the value—produced more reproducible peak areas and greater linearity. A series of standards was then analyzed to generate a calibration curve from 0.5-5000 nM. Peak areas were processed using both x-axis position (FIG. 11A) and x-axis step size (FIG. 11B) of reference-corrected beads. Both calibration curves showed good linearity over the dynamic range, which demonstrates that sugars can be measured over at least four orders of magnitude in concentration. RSD values for each calibration standard varied from 4-12% (n=3 replicates). In both the position and step size data sets, the 0.5 nM standard exhibited higher signal than the blank but the difference did not reach statistical significance (p=0.07). The LODs were determined to be 0.63 nM and 0.52 nM, respectively, for the position and step size curves. In both cases, the blank exhibited an unexpectedly low signal, which caused a slight shift from linearity. However, R2 values were still high in both position and step size curves, with values of 0.9999 and 0.9986, respectively (Table 2). These results indicate that both position and step size are viable for analyte quantitation, but that step size offers a slight advantage in LOD.

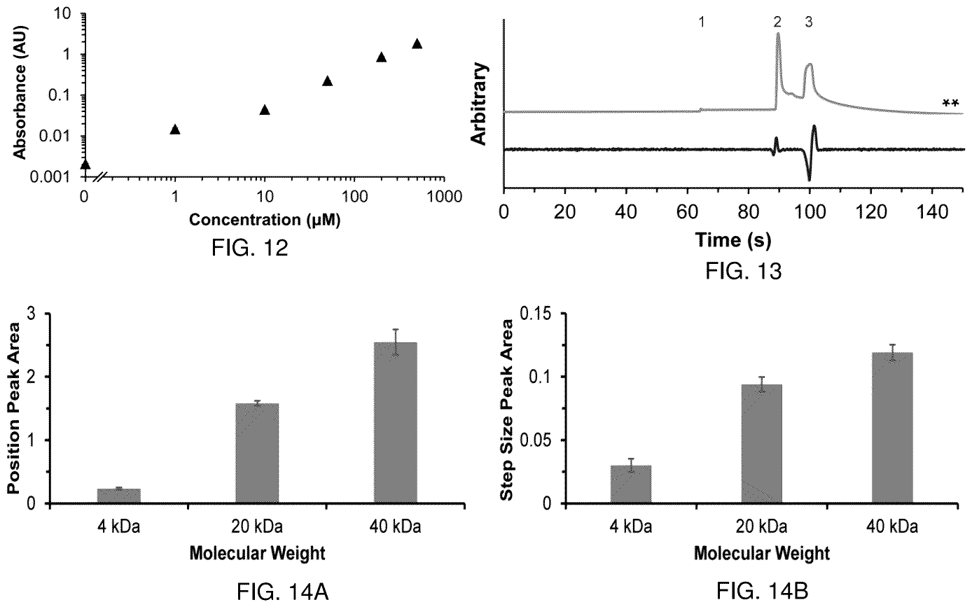

To benchmark the performance of TGE-PMT, a calibration curve using UV-Vis absorbance was also collected. Although sugars generally have weak absorbance, the polysaccharide used in these studies contained >100 monomers, which increased the absorbance of the chain. The dynamic range with absorbance was evaluated from 1-500 mM, and the LOD was determined to be 150 nM (FIG. 12). These results demonstrate TGE-PMT achieved a 300-fold superior concentration LOD compared to the gold-standard technique. Additionally, the 10⁴-fold lower sample volume per analysis in microfluidic TGE than a cuvette-based absorbance analysis culminated in a 4×10⁶-fold superior mass LOD. These findings demonstrate the viability of using bead motion to measure sugars with high sensitivity and low sample volume requirements to achieve superior performance to the conventional analysis technique. TGE-PMT also has the distinct advantage of conducting a separation to resolve the target analyte from contaminants in the formulation, which cannot be accomplished in static absorbance measurements.

TABLE 2
Trendline equations for the calibration
data sets. All concentrations are in nM.

| Technique | Linear Trendline Equation | R² Value |
|---|---|---|
| TGE-PMT Position | y = 0.00302x + 0.1636 | 0.9999 |
| TGE-PMT Step Size | y = 0.000247x + 0.0756 | 0.9986 |
| UV-Vis Absorbance | y = 0.00000374x + 0.0299 | 0.9952 |

### Characterizing Effects of Sugar Molecular Weight

The studies above used 20 kDa CM-dextran as the model analyte. To further investigate factors that govern the detection mechanism of TGE-PMT, sugars over a broader range of molecular weights were analyzed to determine its influence on bead response. Three FITC-CM-dextrans with molecular weights of 4 kDa, 20 kDa, and 40 kDa were measured independently at identical 500 nM concentrations. Peak areas from the resulting measurements were found to increase with increasing molecular weights of the sugars, despite each having the same molar concentration (FIGS. 14A-14B). Trends were similar for both position and step size data processing, although position data had higher sensitivity. The 40 kDa sugar produced a 1.6-fold larger response than the 20 kDa sugar; the 20 kDa sugar produced a 6.7-fold larger response than the 4 kDa sugar. These non-linear responses suggest that PMT is a mass-sensitive detection mode where higher molecular weight sugars perturb bead motion to a greater extent than smaller sugars. However, diminishing increases are realized once the sugars reach a certain molecular weight. This behavior will be investigated in future studies to characterize sugars over a broader mass range.

To further validate that the molecular weight of the sugar influences the bead response, separations were conducted to measure two sugars in a mixed sample. The 4 kDa and 40 kDa FITC-carboxymethyl dextrans were combined and analyzed by TGE. The control analysis directly measuring the fluorescent label revealed three peaks (FIG. 13, **). A low-intensity peak attributed to hydrolyzed dye migrated first (Peak 1), which is consistent with previous TGE studies using fluorescently labeled analytes (see, e.g., patent publication no. US 2023-0314370 A1). Two large peaks then followed (Peaks 2 and 3)—both of which exhibited significant tailing—that were determined to be the 4 kDa and 40 kDa sugars, respectively. Although baseline resolution was not obtained, a clear separation was still observed between the different species. This result is encouraging because it is the first reported separation of polysaccharides by TGE. Although further separation optimizations are warranted to improve resolution, the distribution of molecular weights in the sugar formulations likely contributed to the peak broadening as well. With polysaccharides, the manufacturer states a nominal molecular weight on the product, but in reality, each “single” analyte contains a range of molecular weights. For example, the product sheet for the 4 kDa sugar states that the actual value ranges from 4-6.5 kDa, while the 40 kDa sugar ranges from 40-60 kDa. These broadened mass distributions increase peak widths in TGE electropherograms.

The sugars were then analyzed by TGE-PMT. Two peaks were observed in the resulting electropherograms (FIG. 13, black), with x-axis position and step size data producing similar results. No obvious peak was present for the hydrolyzed dye due to a combination of its low abundance and low molecular weight. Both factors make it difficult to detect with PMT. However, two large peaks were observed that had similar migration times to the sugars in the direct fluorescence control analysis. The peak for the 4 kDa sugar exhibited the characteristic upstream-downstream-upstream segments observed in previous characterizations of the 20 kDa sugar. A flat baseline then followed until a large peak for the 40 kDa sugar migrated. Beads were propelled much farther upstream when this large sugar collided than with the 4 kDa sugar. This is consistent with the data in FIG. 14A-14B where larger PMT responses were observed for larger sugars. Beads then travelled downstream and subsequently back upstream, exhibiting the characteristic peak profile. Peak widths were narrower in PMT than those observed with direct fluorescence, which indicates that beads sense the centers of migrating sugar bands but are insensitive to the low analyte concentrations in the extended peak tailing regions. However, these results demonstrate the utility of the TGE-PMT separations-based sensor to characterize polysaccharide samples in a simple, rapid analysis.

### CONCLUSIONS

Separations-based sensors represent the next stage of sensing technology. By coupling a front-end separation with a back-end sensing scheme, benefits of both platforms can be realized. This report signifies a critical step in this direction. The inline enrichment and separation afforded by TGE (e.g., as described in US Patent Application Publication US 2023-0314370 A1, and Cornejo & Linz, Analytical Chemistry, 19 (14): 5674-5681, 2022) was interfaced with the sensitive label-free detection of PMT. The studies herein defined operating conditions amenable for TGE-PMT and elucidated best practices for data processing. This enabled the analysis of a biomedically relevant sugar over a 10⁴-fold dynamic range and provided a LOD of 0.52 nM. This great analytical performance was achieved without the need to fluorescently label the analyte, thereby decreasing the time and cost of analysis. The separations front-end also identified a likely contaminant peak, which illustrates the potential of the technique for quality control measurements. Studies to provide insight into the detection mechanism revealed that analyte molecular weight influences the bead response, as larger analytes perturb beads to higher degrees. Collectively, the studies in this report lay the foundation for subsequent analyses of sugars with high medical and pharmaceutical significance. TGE-PMT also has the potential to be translated to the detection of other biological macromolecules (e.g., proteins, nucleic acids).

### REFERENCES

(1) Longjohn & Christian, Characterizing Extracellular Vesicles Using Nanoparticle-Tracking Analysis. In Cancer Cell Biology: Methods and Protocols, Christian, S. L. Ed.; Springer US, 2022; pp 353-373.
(2) Tian et al., A Comprehensive Evaluation of Nanoparticle Tracking Analysis (NanoSight) for Characterization of Proteinaceous Submicron Particles. J. Pharm. Sci. 2016, 105 (11), 3366-3375. DOI: 10.1016/j.xphs.2016.08.009.
(3) Vestad et al., Size and concentration analyses of extracellular vesicles by nanoparticle tracking analysis: a variation study. J. Extracell. Vesicles 2017, 6 (1), 1344087. DOI: 10.1080/20013078.2017.1344087.
(4) Maas et al., Possibilities and limitations of current technologies for quantification of biological extracellular vesicles and synthetic mimics. J. Control. Release 2015, 200, 87-96. DOI: 10.1016/j.jconrel.2014.12.041 PMC.
(5) Buskermolen et al., Continuous biomarker monitoring with single molecule resolution by measuring free particle motion. Nat. Commun. 2022, 13 (1), 6052. DOI: 10.1038/s41467-022-33487-3.
(6) Vu et al., Real-Time Immunosensor for Small-Molecule Monitoring in Industrial Food Processes. Anal. Chem. 2023, 95 (20), 7950-7959. DOI: 10.1021/acs.analchem.3c00628.
(7) Bergkamp et al., Real-time continuous monitoring of dynamic concentration profiles studied with biosensing by particle motion. Lab Chip 2023, 23 (20), 4600-4609, 10.1039/D3LC00410D. DOI: 10.1039/D3LC00410D.
(8) Buskermolen et al., Towards continuous monitoring of TNF-α at picomolar concentrations using biosensing by particle motion. Biosens. Bioelectron. 2024, 249, 115934. DOI: doi.org/10.1016/j.bios.2023.115934.
(9) Scott et al., Development of an on-animal separation-based sensor for monitoring drug metabolism in freely roaming sheep. Analyst 2015, 140 (11), 3820-3829, 10.1039/C4AN01928H. DOI: 10.1039/C4AN01928H.
(10) Gunawardhana et al., Progress toward the development of a microchip electrophoresis separation-based sensor with electrochemical detection for on-line in vivo monitoring of catecholamines. Analyst 2020, 145 (5), 1768-1776, 10.1039/C9AN01980D. DOI: 10.1039/C9AN01980D.
(11) Bezerra et al., Nucleic-Acid Driven Cooperative Bioassays Using Probe Proximity or Split-Probe Techniques. Anal. Chem. 2021, 93 (1), 198-214. DOI: 10.1021/acs.analchem.0c04364.
(12) Gonçalves, Fluorescent Labeling of Biomolecules with Organic Probes. Chem. Rev. 2009, 109 (1), 190-212. DOI: 10.1021/cr0783840.
(13) Ruhaak et al., Glycan labeling strategies and their use in identification and quantification. Anal. Bioanal. Chem. 2010, 397 (8), 3457-3481. DOI: 10.1007/s00216-010-3532-z.
(14) Krenkova et al., Comparison of oligosaccharide labeling employing reductive amination and hydrazone formation chemistries. Electrophoresis 2020, 41 (9), 684-690. DOI: doi.org/10.1002/elps.201900475.
(15) Archer-Hartmann et al., Microscale Exoglycosidase Processing and Lectin Capture of Glycans with Phospholipid Assisted Capillary Electrophoresis Separations. Anal. Chem. 2011, 83 (7), 2740-2747. DOI: 10.1021/ac103362r.
(16) Váradi et al., Rapid Magnetic Bead Based Sample Preparation for Automated and High Throughput N-Glycan Analysis of Therapeutic Antibodies. Anal. Chem. 2014, 86 (12), 5682-5687. DOI: 10.1021/ac501573g.
(17) Ward et al., A review of electrophoretic separations in temperature-responsive Pluronic thermal gels. Anal. Chim. Acta 2023, 1276, 341613. DOI: doi.org/10.1016/j.aca.2023.341613.
(18) Kushan & Senses, Thermoresponsive and Injectable Composite Hydrogels of Cellulose Nanocrystals and Pluronic F127. ACS Appl. Bio Mater. 2021, 4 (4), 3507-3517. DOI: 10.1021/acsabm. 1c00046.
(19) Peli Thanthri & Linz, Controlling the separation of native proteins with temperature in thermal gel transient isotachophoresis. Anal. Bioanal. Chem. 2023, 415 (18), 4163-4172. DOI: 10.1007/s00216-022-04331-w.
(20) Cornejo & Linz, Multiplexed miRNA Quantitation Using Injectionless Microfluidic Thermal Gel Electrophoresis. Anal. Chem. 2022, 94 (14), 5674-5681. DOI: 10.1021/acs.analchem.2c00356.
(21) Cornejo & Linz, Selective miRNA quantitation with high-temperature thermal gel electrophoresis. Anal. Chim. Acta 2023, 1275, 341605. DOI: doi.org/10.1016/j.aca.2023.341605.
(22) Peli Thanthri et al., Simultaneous Preconcentration and Separation of Native Protein Variants Using Thermal Gel Electrophoresis. Anal. Chem. 2020, 92 (9), 6741-6747. DOI: 10.1021/acs.analchem.0c00876.
(23) Cornejo & Linz, Harnessing Joule heating in microfluidic thermal gel electrophoresis to create reversible barriers for cell enrichment. Electrophoresis 2021, 42 (11), 1238-1246. DOI: doi.org/10.1002/elps.202000379.
(24) Ward & Linz, Characterizing the impact of thermal gels on isotachophoresis in microfluidic devices. Electrophoresis 2020, 41 (9), 691-696. DOI: 10.1002/elps.201900407.
(25) Schindelin et al., Fiji: an open-source platform for biological-image analysis. Nat. Methods 2012, 9 (7), 676-682. DOI: 10.1038/nmeth.2019.
(26) Ershov et al., TrackMate 7: integrating state-of-the-art segmentation algorithms into tracking pipelines. Nat. Methods 2022, 19 (7), 829-832. DOI: 10.1038/s41592-022-01507-1.
(27) Jia et al., The time, size, viscosity, and temperature dependence of the Brownian motion of polystyrene microspheres. Am. J. Phys. 2007, 75 (2), 111-115. DOI: 10.1119/1.2386163.
(28) Vaz et al., Chromophoreasy, an Excel-Based Program for Detection and Integration of Peaks from Chromatographic and Electromigration Techniques. J. Brazil Chem. Soc. 2016, 27, 1899-1911.
(29) Petrovici et al., Dextran Formulations as Effective Delivery Systems of Therapeutic Agents. Molecules 2023, 28 (3), 1086.
(30) Ning et al., Carboxymethyl dextran-coated liposomes: Toward a robust drug delivery platform. Soft Matter 2011, 7 (19), 9394-9401, 10.1039/C1SM05814B. DOI: 10.1039/C1SM05814B.
(31) Devlin et al., Tools for the Quality Control of Pharmaceutical Heparin. Medicina 2019, 55 (10), 636.
(32) Zhang et al., Reversible thermo-responsive sieving matrix for oligonucleotide separation. Lab Chip 2006, 6 (4), 526-533, 10.1039/B515557F. DOI: 10.1039/B515557F.
(33) Starchev et al., Brownian Motion and Electrophoretic Transport in Agarose Gels Studied by Epifluorescence Microscopy and Single Particle Tracking Analysis. J. Phys. Chem. B 1997, 101 (29), 5659-5663. DOI: 10.1021/jp970725y.
(34) Karslake et al., SMAUG: Analyzing single-molecule tracks with nonparametric Bayesian statistics. Methods 2021, 193, 16-26. DOI: doi.org/10.1016/j.ymeth.2020.03.008.
(35) Mitra et al., Microchip electrophoresis at elevated temperatures and high separation field strengths. Electrophoresis 2014, 35 (2-3), 374-378. DOI: 10.1002/elps.201300427.
(36) Wu et al., Viscosity-adjustable block copolymer for DNA separation by capillary electrophoresis. Electrophoresis 1998, 19 (2), 231-241. DOI: 10.1002/elps. 1150190216.
(37) Exploring Our Fluid Earth, Wave Energy and Wave Changes with Depth. University of Hawai'i, 1999. manoa.hawaii.edu/exploringourfluidearth/physical/waves/wave-energy-and-wave-changes-depth.
International Patent Publication No. WO2020/104814A2
U.S. Pat. No. 10,301,041
US Patent Application Publication No. 2012/0288852A1
Zong et al., Single-Molecule Level Rare Events Revealed by Dynamic Surface-Enhanced Raman Spectroscopy, Analytical Chemistry, 92 (24): 15806-15801, 2020; doi.org/10.1021/acs.analchem.0c02936
Scott & Carron, Dynamic Surface Enhanced Raman Spectroscopy (SERS): Extracting SERS from Normal Raman Scattering, Analytical Chemistry, 85 (20): 8448-8451, 2012; doi.org/10.1021/ac301914a

### Example 3: Label-Free Peptide Analysis

In addition to the polysaccharide example above, analysis methods have also been developed for peptides. This embodiment is important because of the high pharmaceutical significance of peptides.

Simply synthesizing therapeutic peptides with the correct linear sequence of amino acids is insufficient to validate drug structure. Peptides can adopt higher order conformations that dictate their biological activity. Disulfide bonds, for example, are S—S linkages that impact the tertiary structure of peptides and are thus critical in controlling peptide function. A common problem, however, is the scrambling (mismatching) of two or more disulfide bonds, where disulfide bonds transition between pairs of sulfur atoms. The polarities and masses of native and scrambled peptides are generally unchanged, so LC-MS cannot characterize changes in this higher order structure. The provided method was developed to address this critical problem by integrating peptide characterizations into the TGE-PMT analysis platform.

An analogous microfluidic scheme to the one reported above was employed for this study. Peptide and fluorescent nanoparticles were added to a stock of thermal gel, which was then loaded throughout single-channel microfluidic devices. An inline enrichment and separation was conducted to analyze the sample components. More specifically, single-channel microfluidic devices were fabricated with reference beads outside the channel, similarly to Example 2. The channel was then loaded with the peptide sample in a thermal gel comprised of 30% Pluronic F127 in 20 mM tris-HCl. The cathode reservoir was filled with a solution containing 800 mM glycine and 5 mM tris-HCl. The anode reservoir was filled with a solution containing 5 mM tris-HCl, 10 mM ammonium acetate, and 30% Pluronic F127.

Data was then processed using a procedure distinct from that used with the polysaccharides. For this peptide analysis, all beads in a region of interest (ROI) were treated as a single entity, and the average position of the ensemble was tracked frame-to-frame. This same approach was applied to two additional ROIs in the field of view. Signal from all three ROIs was summed after including time delays for the analyte band to reach each successive ROI. By including more beads in each ROI and averaging multiple ROIs together, sensitivity of the analysis was significantly enhanced compared to the previous single-bead tracking experiments.

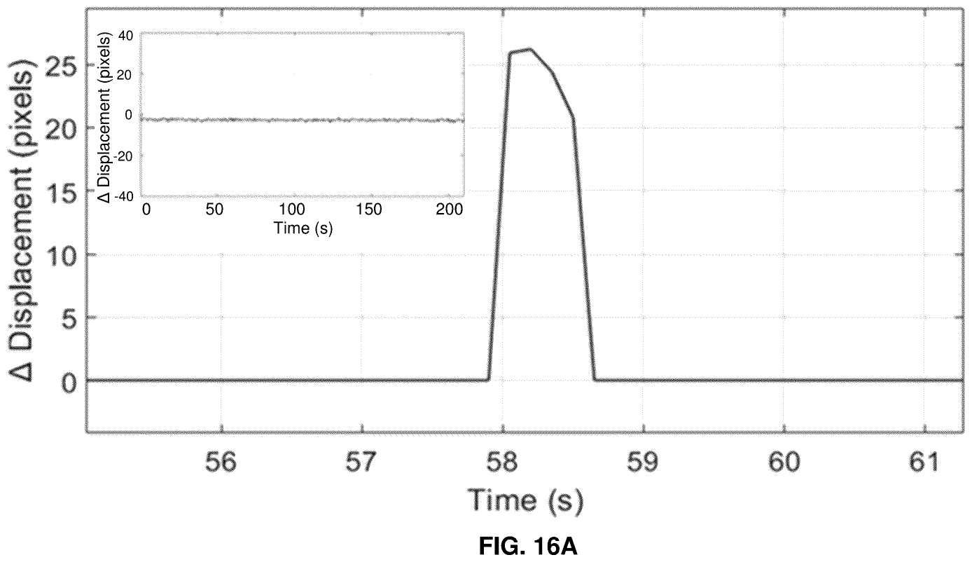

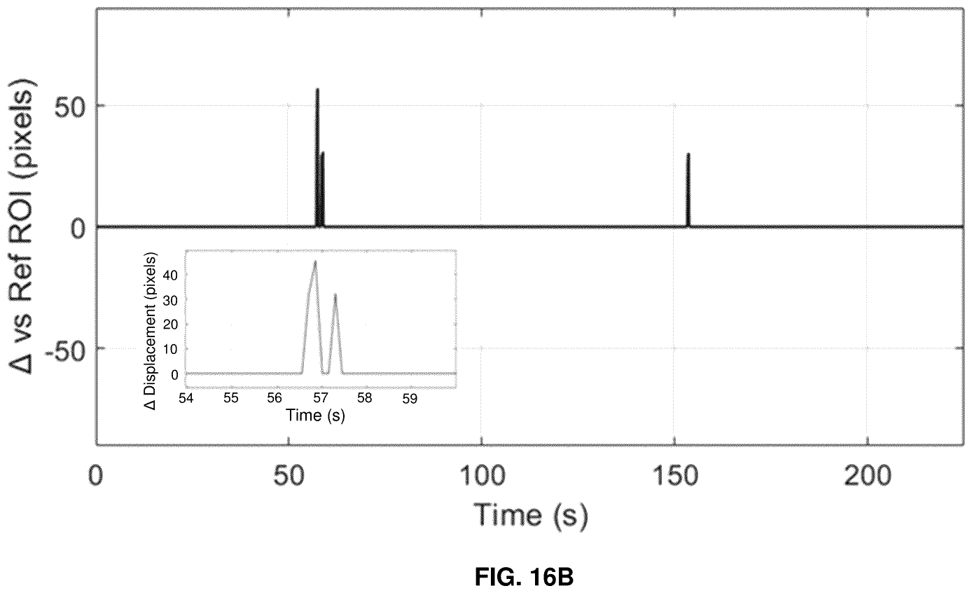

Insulin was selected as a model peptide, because it possesses three disulfide bonds: two between the A chain and B chain and one within the A chain. Molecular weights of the intact native peptide, A chain, and B chain are 5.8 kDa, 2.3 kDa and 3.5 kDa, respectively. Initial analyses showed that native insulin produced a single peak (FIG. 16A). No peaks were observed in negative controls (FIG. 16A, inset), demonstrating good selectivity for the peptide. A calibration series was then analyzed where insulin was prepared from 0.5-50 mM in 10-fold increments. A linear response was observed across the range, indicating the quantitative linearity of TGE-PMT for peptide measurements.

To begin evaluating disulfide bonded conformations, insulin was incubated with dithiothreitol to reduce the disulfide bonds. No alkylating agent was used during this sample preparation to enable scrambled disulfide bonds to form. Analysis of this sample by TGE-PMT revealed three peaks: two that migrated early, one that migrated late (FIG. 16B). Tentative peak assignments suggest that the first two peaks are unique conformations of the A chain (2.3 kDa) whereas the third peak is the dissociated B chain (3.5 kDa). These results illustrate that the provided peptide TGE-PMT analysis can distinguish protein confirmation, including where the primary sequence of the peptide(s) are identical. These results are encouraging because multiple distinct peptides and peptide conformations can be detected using the analytical scheme while employing a label-free detection format.

### (XV) Closing Paragraphs

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. A material effect would cause a statistically significant alteration (e.g., reduction or increase) in detection and/or quantification of biomolecule(s) undergoing separation.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

1. An improved method of analyte detection and/or quantification in gel electrophoresis, the improvement comprising:
including tracking particles within the gel; and
indirectly detecting a wavefront of analyte migrating within the gel, which migrating is caused at least in part by analyte interaction with an applied electric field, the indirect detecting comprising detecting movement of the tracking particles in response to collisions with analyte molecules in the wavefront and/or deformations of the gel.

2. The method of claim 1, wherein the tracking particles are characterized as one or more of:
incapable of interacting with the analyte by binding, adsorption, or chemical reaction;
have an average diameter less than 10 microns;
have an average diameter selected from: ≤8 microns, ≤5 microns, ≤2 microns, ≤1 micron, ≤900 nanometers, ≤800 nanometers, ≤750 nanometers, ≤700 nanometers, ≤600 nanometers, ≤500 nanometers, ≤400 nanometers, ≤300 nanometers, ≤200 nanometers, ≤100 nanometers, ≤80 nanometers, ≤75 nanometers, ≤50 nanometers, ≤40 nanometers, 100 nanometers to 2 microns, 100 nanometers to 1 micron, 100-800 nanometers, 100-600 nanometers, 100-500 nanometers, 100-400 nanometers, 100-300 nanometers, 100-250 nanometers, 150-300 nanometers, 150-250 nanometers, 200-300 nanometers, 50-300 nanometers, 50-250 nanometers, 50-300 nanometers, 50-250 nanometers, 15-300 nanometers, 15-250 nanometers, 15-300 nanometers, 15-250 nanometers, 15 nanometers, 20 nanometers, 25 nanometers, 40 nanometers, 50 nanometers, 75 nanometers, 100 nanometers, 150 nanometers, 200 nanometers, 250 nanometers, 300 nanometers, 350 nanometers, 400 nanometers, 500 nanometers, 600 nanometers, 750 nanometers, 800 nanometers, 1 micron, or 2 microns; and/or
include at least two different defined-size tracking particle populations.

3-5. (canceled)

6. The method of claim 1, wherein detecting movement of the tracking particles comprises determining displacement in an X dimension, parallel to the direction of migration of the analyte wavefront;
of at least one tracking particle from a time zero position; and/or
from a time zero position of a plurality of tracking particles within a region of interest (ROI).

7. The method of claim 6, further comprising:
calculating tracking particle displacement based on step size analysis comprising analysis from one image frame to the next; and/or
determining the displacement of more than one, at least three, at least five, at least seven, at least ten, at least 20, at least 50, at least 75, at least 100, at least 1000, at least 10,000, or more than 10,000 tracking particles.

8-9. (canceled)

10. The method of claim 1, comprising indirectly detecting migration of more than one analyte, each analyte having a wavefront of migration distinguishable at least in part from the wavefront of other analyte(s) migrating in the gel.

11. A method of label-free injectionless gel electrophoresis, comprising:
providing a microfluidic device having a channel, the channel having a first end and a second end, the microfluidic device having a first reservoir coupled to the first end of the channel and a second reservoir coupled to the second end of the channel;
loading into the channel of the microfluidic device a substantially homogenous mixture comprising:
a mixed analyte sample;
a plurality of tracking particles; and
a gel solution;
providing a first reservoir solution in the first reservoir;
providing a second reservoir solution in the second reservoir; and
applying an electric field across the microfluidic device.

12. The method of claim 11, wherein one or more of:
the tracking particles comprise nanoparticles, microparticles, or quantum dots;
the tracking particles comprise one or more of a polymer, a metal, silica, glass, a magnetic material, or a lipid;
the tracking particles comprise a polymer comprising a dextran, a polystyrene, an acrylate, a poly(ethylene glycol) (PEG), a peptide, or a nucleic acid;
the tracking particles comprise a detectable label;
the first reservoir solution includes a first electrolyte and the second reservoir solution includes a second electrolyte;
the mixed analytes comprise biomolecules;
the mixed analyte sample comprises at least one of nucleic acids, carbohydrates, peptides, or proteins;
the gel is a sieving gel for resolving analytes in the sample;
the gel is thermally responsive;
the gel comprises one or more of: agarose, polyacrylamide, a polymer sieving matrix, a capillary gel electrophoresis matrix, or a thermal gel polymer;
the gel comprises a thermal gel polymer comprising one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA); or
reference particles are embedded in at least a portion of the microfluidic device adjacent to the channel.

13-16. (canceled)

17. The method of claim 11, wherein the microfluidic device further comprises a first electrode arranged in the first reservoir and a second electrode arranged in the second reservoir.

18. The method of claim 17, wherein method comprises;
anionic analytes migrating from the first reservoir to the second reservoir, and:
the first electrode is a cathodic electrode;
the first reservoir solution is a cathodic reservoir solution;
the first reservoir is a cathodic reservoir;
the second electrode is an anodic electrode;
the second reservoir solution is an anodic reservoir solution; and
the second reservoir is an anodic reservoir;
or
cationic analytes migrating from the first reservoir to the second reservoir, and:
the first electrode is an anodic electrode;
the first reservoir solution is an anodic reservoir solution;
the first reservoir is an anodic reservoir;
the second electrode is a cathodic electrode;
the second reservoir solution is a cathodic reservoir solution; and
the second reservoir is a cathodic reservoir.

19-21. (canceled)

22. The method of claim 11, further comprising one or more of:
solidifying the gel solution;
including buffer in the gel solution and/or the mixed analyte sample; or
detecting separation of analytes of the mixed analyte sample in the channel.

23-29. (canceled)

30. The method of claim 11, comprising indirectly detecting a wavefront of analyte migrating within the gel by detecting movement of the tracking particles in response to collisions of analyte molecules in the wavefront with the tracking particles and/or deformations of the gel, wherein the analyte migrating results at least in part from analyte interaction with:
an electric field:
an applied electric current and/or
an applied electric voltage.

31. (canceled)

32. A microfluidic device, comprising:
a channel, configured to accommodate a mixed analyte sample mixed with a gel solution, the channel having a first end and a second end;
embedded within at least a portion of a wall of the channel, reference particles;
a first reservoir coupled to the first end of the channel, the first reservoir being configured to accommodate a first reservoir solution;
a second reservoir coupled to the second of the channel, the second reservoir being configured to accommodate a second reservoir solution;
a first electrode arranged in the first reservoir; and
a second electrode arranged in the second reservoir;
wherein the first electrode and the second electrode are configured to apply an electric field across the microfluidic device.

33. The device of claim 32, wherein device is configured for:
anionic analytes to migrate from the first reservoir to the second reservoir, and:
the first electrode is a cathodic electrode;
the first reservoir solution is a cathodic reservoir solution;
the first reservoir is a cathodic reservoir;
the second electrode is an anodic electrode;
the second reservoir solution is an anodic reservoir solution; and
the second reservoir is an anodic reservoir;
or
cationic analytes to migrate from the first reservoir to the second reservoir, and:
the first electrode is an anodic electrode;
the first reservoir solution is an anodic reservoir solution;
the first reservoir is an anodic reservoir;
the second electrode is a cathodic electrode;
the second reservoir solution is a cathodic reservoir solution; and
the second reservoir is a cathodic reservoir.

34. (canceled)

35. The device of claim 32, wherein one or more of:
the sample comprises at least one of nucleic acids, carbohydrates, peptides, or proteins;
the gel is a sieving gel for resolving the sample;
the gel is thermally responsive; or
the reference particles are configured as device movement/drift control reference markers.

36-38. (canceled)

39. A method of assaying migration of one or more analytes in a liquid or a gel contained in a device, the method comprising:
contacting one or more migrating analytes with a plurality of tracking particles of average diameter less than 10 microns, the particles being incapable of interacting with the analyte by binding, adsorption, or chemical reaction;
observing motion of some or all of the tracking particles by at least one optical, fluorescence, or other electromagnetic measurement technique; and
using differing motion of the tracking particles to infer presence and/or concentration of the migrating analyte.

40. The method of claim 39, wherein the observing comprises one or more of:
observing one or more of fluorescence, fluorescence lifetime, resonance energy transfer, phosphorescence, reflection, polarization, scattering, absorbance, chemiluminescence, or magnetic properties of some or all of the tracking particles; or
detecting electromagnetic emission(s) of some or all of the tracking particles at more than one wavelength.

41. (canceled)

42. The method of claim 39, further comprising one or more of:
particle tracking, single-particle tracking or tethered-particle motion tracking;
observing the tracking particles by camera, digital camera, PMT, scanner, microscope, telescope, detector array, time-gated, chopped, frequency-modulated, wavelength-filtered, polarization-sensitive, Raman, Surface-enhanced Raman, high numerical aperture, color-sensitive, lifetime, FRET, FRAP, intensified, phosphorescence, resistivity, ellipsometer, or high-density CCD observation, in flow, on a surface, or in suspension;
assaying more than one migrating analyte, which migrating analytes are separated by the migration, and the method comprises using differing motion of the tracking particles to infer presence and/or concentration of the more than one migrating analyte;
assaying migration of one or more analytes in a device comprising reference particles substantially equivalent in size and detection capability to the tracking particles with which the analyte(s) is contacted, wherein the method further comprises controlling for device movement when calculating the differing motion of the tracking particles by detecting movement of one or more reference particles; or
embedding or otherwise attaching reference particles in/on the device, which reference particles are used to correct for device drift and/or to improve signal quality of movement of tracking particles used in the method.

43. The method of claim 39, in which the migrating of one or more analytes in the liquid or gel comprises one or more of: electrophoretic, dielectrophoretic, isotachophoretic, or sedimentation motion.

44-45. (canceled)

46. The method of claim 39, wherein the liquid is contained at least in part within a solidified gel, and one or more of:
the solidified gel is a sieving gel for resolving migrating analytes;
the solidified gel is thermally responsive;
the solidified gel comprises one or more of: agarose, poly(Original) acrylamide, a polymer sieving matrix, a capillary gel electrophoresis matrix, or a thermal gel polymer; or
the solidified gel comprises a thermal gel polymer comprising one or more of: Pluronic F-127 (PF-127; aka Poloxamer 407), Pluronic F-68, dimyristoyl-sn-glycero-3-phosphocholine, 1,2-dihexanoyl-sn-glycero-3-phosphocholine, poly(N-isopropylacrylamide)-g-poly(ethyleneoxide), N,N′-dimethylacrylamide (DMA) and N,N′-diethylacrylamide (DEA), N-ethoxyethylacrylamide (NEEA), or N-methoxyethylacrylamide (NMEA).

47-50. (canceled)

51. The method of claim 39, wherein the tracking particles comprise one or more of:
nanoparticles, microparticles, or quantum dots;
one or more of a polymer, a metal, silica, glass, a magnetic material, or a lipid; or
a polymer comprising a dextran, a polystyrene, an acrylate, a poly(ethylene glycol), a peptide, or a nucleic acid.

52-53. (canceled)

54. The method of claim 39, wherein:
a temperature of one or more elements of a system comprising the device is controlled;
the liquid is contained at least in part within a solidified gel; or
both.

55-59. (canceled)
